(19) United States
(12) Patent Application Publication
(10) Pub. No.: US 2023/0392207 A1
(43) Pub. Date: Dec. 7, 2023

(54) CIRCULATING RNA BIOMARKERS FOR PREECLAMPSIA

(71) Applicant: ILLUMINA, INC., San Diego, CA (US)

(72) Inventors: Sarah E. Shultzaberger, San Diego, CA (US); Suzanne Rohrback, San Diego, CA (US); Carlo Randise-Hinchliff, San Diego, CA (US)

(73) Assignee: ILLUMINA, INC., San Diego, CA (US)

(21) Appl. No.: 18/203,734

(22) Filed: May 31, 2023

Related U.S. Application Data
(60) Provisional application No. 63/348,846, filed on Jun. 3, 2022.

(51) Int. Cl. C12Q 1/6883; C12Q 1/6806

(52) U.S. Cl. CPC C12Q 1/6883; C12Q 1/6806; C12Q 2600/158

(57) ABSTRACT

The present invention includes methods and materials for use in the detection preeclampsia and/or determining an increased risk for preeclampsia in a pregnant female, the method including identifying in a biosample obtained from the pregnant women circulating RNA (C-RNA) molecules associated with preeclampsia.

### CONTINUING APPLICATION DATA

This application claims the benefit of U.S. Provisional Application Ser. No. 63/348,846, filed Jun. 3, 2022, which is incorporated by reference herein.

### FIELD OF INVENTION

The present invention relates generally to methods and materials for use in the detection and early risk assessment for the pregnancy complication preeclampsia.

### BACKGROUND

Preeclampsia is a condition that occurs only during pregnancy, affecting 5% to 8% of all pregnancies. It is the direct cause of 10%-15% of maternal deaths and 40% of fetal deaths. The three main symptoms of preeclampsia may include high blood pressure, swelling of hands and feet, and excess protein in the urine (proteinuria), occurring after week 20 of pregnancy. Other signs and symptoms of preeclampsia may include severe headaches, changes in vision (including temporary loss of vision, blurred vision, or light sensitivity), nausea or vomiting, decreased urine output, decreased platelets levels (thrombocytopenia), impaired liver function, and shortness of breath, caused by fluid in the lung. See, for example Steegers et al., 2010, Lancet; 376:631-644. doi: 10.1016/S0140-6736(10)60279-6; and Miller et al., 2008, Semin Perinatol; 32:274-280. doi: 10.1053/j.semperi.2008.04.010.

The more severe the preeclampsia and the earlier it occurs in pregnancy, the greater the risks for mother and baby. Preeclampsia may require induced labor and delivery or delivery by cesarean delivery. Left untreated, preeclampsia can lead to serious, even fatal, complications for both the mother and baby. Complications of preeclampsia include fetal growth restriction FGR), low birth weight, preterm birth, placental abruption, HELLP syndrome (hemolysis, elevated liver enzymes, and low platelet count syndrome), eclampsia (a severe form of preeclampsia that leads to seizures), organ damage, including kidney, liver, lung, heart, eye damage, stroke, or other brain injury. See, for example, “Preeclampsia—Symptoms and causes—Mayo Clinic,” Apr. 15, 2022, available at on the worldwide web at mayoclinic.org/diseases-conditions/preeclampsia/symptoms-causes/syc-20355745.

With early detection and treatment, most women can deliver a healthy baby if preeclampsia is detected early and treated with regular prenatal care. Although various protein biomarkers display changed levels in maternal serum at presymptomatic stages, these biomarkers lack discriminative and predictive power in individual patients (Karumanchi and Granger, 2016, Hypertension; 67(2): 238-242). Thus, the identification of biomarkers for the early detection of preeclampsia is critical for the early diagnosis and treatment of preeclampsia.

### SUMMARY OF THE INVENTION

The present invention includes a method of detecting preeclampsia and/or determining an increased risk for preeclampsia in a pregnant female, the method comprising:
identifying in a biosample obtained from the pregnant female a circulating RNA (C-RNA) molecule encoding at least a portion of a protein selected from any one or more, any two or more, any three or more, or all four of HCG4P8, EYS, AGGF1P10, and GLYATL2;
wherein identifying the C-RNA molecule encoding at least a portion of a protein selected from any one or more, any two or more, any three or more, or all four of HCG4P8, EYS, AGGF1P10, and GLYATL2 is indicative of preeclampsia and/or an increased risk for preeclampsia in the pregnant female.

The present invention includes a method of detecting preeclampsia and/or determining an increased risk for preeclampsia in a pregnant female, the method comprising:
purifying a population of circulating RNA (C-RNA) molecules from a biosample obtained from the pregnant female;
identifying protein coding sequences encoded by the C-RNA molecules within the purified population of C-RNA molecules;
wherein the identification of protein coding sequences encoded by the C-RNA molecules encoding at least a portion of a protein are selected from any one or more, any two or more, any three or more, or all four of HCG4P8, EYS, AGGF1P10, and GLYATL2 is indicative of preeclampsia and/or an increased risk for preeclampsia in the pregnant women.

In some aspects, identifying protein coding sequences encoded by the C-RNA molecules within the biosample includes hybridization, reverse transcriptase PCR, microarray chip analysis, or sequencing. In some aspects, sequencing includes clonal amplification and massively parallel sequencing of clonally amplified molecules. In some aspects, sequencing includes RNA sequencing.

In some aspects of a method of detecting preeclampsia and/or determining an increased risk for preeclampsia in a pregnant female, the method further includes:
removing intact cells from the biosample;
treating the biosample with a deoxynuclease (DNase) to remove cell free DNA (cfDNA;
synthesizing complementary DNA (cDNA) from C-RNA molecules in the biosample; and/or
enriching the cDNA sequences for sequences that encode proteins;
prior to identifying protein coding sequence encoded by the circulating RNA (C-RNA) molecules.

The present invention includes a method of detecting preeclampsia and/or determining an increased risk for preeclampsia in a pregnant female, the method comprising:
removing intact cells from a biosample obtained from the pregnant female; treating the biosample with a deoxynuclease (DNase) to remove cell free DNA (cfDNA);
synthesizing complementary DNA (cDNA) from RNA molecules in the biosample;
enriching the cDNA sequences for DNA sequences that encode proteins;
sequencing the resulting enriched cDNA sequences; and
identifying protein coding sequences encoded by enriched cDNA sequences;
wherein the identification of protein coding sequences encoded by the enriched cDNA sequences encoding at least a portion of a protein selected from any one or more, any two or more, any three or more, or all four of HCG4P8, EYS, AGGF1P10, and GLYATL2 is indicative of preeclampsia and/or an increased risk for preeclampsia in the pregnant women.

In some aspects, detecting preeclampsia and/or determining an increased risk for preeclampsia comprises detecting early preeclampsia and/or determining an increased risk for early preeclampsia.

The present invention includes a method comprising:
removing intact cells from a biosample obtained from a pregnant female;
treating the biosample with a deoxynuclease (DNase) to remove cell free DNA (cfDNA);
synthesizing complementary DNA (cDNA) sequences from RNA molecules in the biosample;
enriching the cDNA sequences for DNA sequences that encode proteins;
sequencing the resulting enriched cDNA sequences; and
identifying within the resulting protein coding sequences encoded by the enriched C-RNA molecules protein coding sequences including at least a portion of a protein selected from any one or more, any two or more, any three or more, or all four of HCG4P8, EYS, AGGF1P10, and GLYATL2.

In some aspects of a method of the present invention, the biosample is obtained from a pregnant female at about 9 weeks gestation to about 12 weeks gestation, about 13 weeks gestation to about 16 weeks gestation, about 17 weeks gestation to about 20 weeks gestation, about 21 weeks gestation to about 24 weeks gestation, about 25 weeks gestation to about 28 weeks gestation, about 29 weeks gestation to about 32 weeks gestation, about 33 weeks gestation to about 36 weeks gestation, or about 37 weeks gestation to about 40 weeks gestations.

In some aspects of a method of the present invention, the biosample is obtained from a pregnant female at about 12 weeks gestation, about 20 weeks gestations, about 28 weeks gestation, or about 36 weeks gestation.

In some aspects of a method of the present invention, the biosample comprises plasma.

In some aspects of a method of the present invention, the sample is a blood sample, and the blood sample is:
not exposed to EDTA prior to processing the blood sample into plasma;
processed into plasma within about 24 to about 72 hours of the blood draw;
maintained, stored, and/or shipped at room temperature prior to processing into plasma; and/or
maintained, stored, and/or shipped without exposure to chilling or freezing prior to processing into plasma.

In some aspects of a method of the present invention: HCG4P8 is downregulated in comparison to a normal control; EYS is downregulated in comparison to a normal control; AGGF1P10 is downregulated in comparison to a normal control; and/or GLYATL2 is upregulated in comparison to a normal control.

In some aspects of a method of the present invention, the method further includes identifying within the biosample a circulating RNA (C-RNA) molecule encoding at least a portion of a protein selected from:
(a) any one or more, any two or more, any three or more, any four or more, any five or more, any six or more, any seven or more, any eight or more, any nine or more, any ten or more, any eleven or more, any twelve, any thirteen or more, any fourteen or more, any fifteen or more, any sixteen or more, any seventeen or more, any eighteen or more, any nineteen or more, any twenty or more, any twenty one or more, any twenty two or more, any twenty three or more, any twenty four or more, any twenty five or more, any fifty or more, any seventy or more, or all seventy-five of ARRDC2, JUN, SKIL, ATP13A3, PDE8B, GSTA3, PAPPA2, TIPARP, LEP, RGP1, USP54, CLEC4C, MRPS35, ARHGEF25, CUX2, HEATR9, FSTL3, DDI2, ZMYM6, ST6GALNAC3, GBP2, NES, ETV3, ADAM17, ATOH8, SLC4A3, TRAF3IP1, TTC21A, HEG1, ASTE1, TMEM108, ENC1, SCAMPI, ARRDC3, SLC26A2, SLIT3, CLIC5, TNFRSF21, PPP1R17, TPST1, GATSL2, SPDYE5, HIPK2, MTRNR2L6, CLCN1, GINS4, CRH, C10orf2, TRUB1, PRG2, ACY3, FAR2, CD63, CKAP4, TPCN1, RNF6, THTPA, FOS, PARN, ORAI3, ELMO3, SMPD3, SERPINF1, TMEM11, PSMD11, EBI3, CLEC4M, CCDC151, CPAMD8, CNFN, LILRA4, ADA, C22orf39, PI4KAP1, and ARFGAP3; or
(b) any one or more, any two or more, any three or more, any four or more, any five or more, any six or more, any seven or more, any eight or more, any nine or more, any ten or more, any eleven or more, any twelve or more, any thirteen or more, any fourteen or more, any fifteen or more, any sixteen or more, any seventeen or more, any eighteen or more, any nineteen or more, any twenty or more, any twenty one or more, any twenty two or more, any twenty three or more, any twenty four or more, any twenty five or more, any twenty six of more, or all twenty-seven of TIMP4, FLG, HTRA4, AMPH, LCN6, CRH, TEAD4, ARMS2, PAPPA2, SEMA3G, ADAMTS1, ALOX15B, SLC9A3R2, TIMP3, IGFBP5, HSPA12B, CLEC4C, KRT5, PRG2, PRX, ARHGEF25, ADAMTS2, DAAM2, FAM107A, LEP, NES, and VSIG4; or
(c) any one or more, any two or more, any three or more, any four or more, any five or more, any six or more, any seven or more, any eight or more, any nine or more, any ten or more, any eleven or more, any twelve, any thirteen or more, any fourteen or more, any fifteen or more, any sixteen or more, any seventeen or more, any eighteen or more, any nineteen or more, any twenty or more, any twenty one or more, any twenty two or more, any twenty three or more, any twenty four or more, any twenty five or more, any fifty or more, any seventy-five or more, any one hundred or more, or all one hundred twenty-two of CYP26B1, IRF6, MYH14, PODXL, PPP1R3C, SH3RF2, TMC7, ZNF366, ADCY1, C6, FAM219A, HAO2, IGIP, IL1R2, NTRK2, SH3PXD2A, SSUH2, SULT2A1, FMO3, FSTL3, GATA5, HTRA1, C8B, H19, MN1, NFE2L1, PRDM16, AP3B2, EMP1, FLNC, STAG3, CPB2, TENC1, RP1L1, A1CF, NPR1, TEK, ERRFIl, ARHGEF15, CD34, RSPO3, ALPK3, SAMD4A, ZCCHC24, LEAP2, MYL2, NRG3, ZBTB16, SERPINA3, AQP7, SRPX, UACA, ANO1, FKBP5, SCN5A, PTPN21, CACNAlC, ERG, SOX17, WWTR1, AIF1L, CA3, HRG, TAT, AQP7P1, ADRA2C, SYNPO, FN1, GPR116, KRT17, AZGP1, BCL6B, KIFIC, CLIC5, GPR4, GJA5, OLAH, C14orf37, ZEB1, JAG2, KIF26A, APOLD1, PNMT, MYOM3, PITPNM3, TIMP4, HTRA4, AMPH, LCN6, CRH, TEAD4, ARMS2, PAPPA2, SEMA3G, ADAMTS1, ALOX15B, SLC9A3R2, TIMP3, IGFBP5, HSPA12B, PRG2, PRX, ARHGEF25, ADAMTS2, DAAM2, FAM107A, LEP, NES, VSIG4, HBG2, CADM2, LAMP5, PTGDR2, NOMO1, NXF3, PLD4, BPIFB3, PACSIN1, CUX2, FLG, CLEC4C, and KRT5; or
(d) any one or more, any two or more, any three or more, any four or more, any five or more, any six or more, any seven or more, any eight or more, any nine or more, any ten or more, any eleven or more, any twelve or more, any thirteen or more, any fourteen or more, any fifteen or more, any sixteen or more, any seventeen or more, any eighteen or more, any nineteen or more, any twenty or more, any twenty-one or more, any twenty-two or more, any twenty-three or more, any twenty-four or more, any twenty-five or more, any twenty-six or more, any twenty-seven or more, any twenty-eight or more, any twenty-nine or more, or all thirty of VSIG4, ADAMTS2, NES, FAM107A, LEP, DAAM2, ARHGEF25, TIMP3, PRX, ALOX15B, HSPA12B, IGFBP5, CLEC4C, SLC9A3R2, ADAMTS1, SEMA3G, KRT5, AMPH, PRG2, PAPPA2, TEAD4, CRH, PITPNM3, TIMP4, PNMT, ZEB1, APOLD1, PLD4, CUX2, and HTRA4; or
(e) any one or more, any two or more, any three or more, any four or more, any five or more, any six or more, any seven or more, any eight or more, any nine or more, any ten or more, any eleven or more, any twelve or more, any thirteen or more, any fourteen or more, any fifteen or more, any sixteen or more, any seventeen or more, any eighteen or more, any nineteen or more, any twenty or more, any twenty-one or more, any twenty-two or more, any twenty-three or more, any twenty-four or more, any twenty-five or more, or all twenty-six of ADAMTS1, ADAMTS2, ALOX15B, AMPH, ARHGEF25, CELF4, DAAM2, FAM107A, HSPA12B, HTRA4, IGFBP5, KCNA5, KRT5, LCN6, LEP, LRRC26, NES, OLAH, PACSIN1, PAPPA2, PRX, PTGDR2, SEMA3G, SLC9A3R2, TIMP3, and VSIG4; or
(f) any one or more, any two or more, any three or more, any four or more, any five or more, any six or more, any seven or more, any eight or more, any nine or more, any ten or more, any eleven or more, any twelve or more, any thirteen or more, any fourteen or more, any fifteen or more, any sixteen or more, any seventeen or more, any eighteen or more, any nineteen or more, any twenty or more, any twenty-one or more, or all twenty-two of ADAMTS1, ADAMTS2, ALOX15B, ARHGEF25, CELF4, DAAM2, FAM107A, HTRA4, IGFBP5, KCNA5, KRT5, LCN6, LEP, LRRC26, NES, OLAH, PRX, PTGDR2, SEMA3G, SLC9A3R2, TIMP3, and VSIG4; or
(g) any one or more, any two or more, any three or more, any four or more, any five or more, any six or more, any seven or more, any eight or more, any nine or more, any ten or more, or all eleven of CLEC4C, ARHGEF25, ADAMTS2, LEP, ARRDC2, SKIL, PAPPA2, VSIG4, ARRDC4, CRH, and NES; or
(h) any one or more, any two or more, any three or more, any four or more, any five or more, any six or more, any seven or more, any eight or more, any nine or more, any ten or more, any eleven or more, any twelve or more, any thirteen or more, any fourteen or more, any fifteen or more, any sixteen or more, any seventeen or more, any eighteen or more, any nineteen or more, any twenty or more, any twenty-one or more, any twenty-two or more, any twenty-three or more, or all twenty-four of LEP, PAPPA2, KCNA5, ADAMTS2, MYOM3, ATP13A3, ARHGEF25, ADA, HTRA4, NES, CRH, ACY3, PLD4, SCT, NOX4, PACSIN1, SERPINF1, SKIL, SEMA3G, TIPARP, LRRC26, PHEX, LILRA4, and PER1; or
(i) any one or more, any two or more, any three or more, any four or more, any five or more, any six or more, any seven or more, any eight or more, any nine or more, any ten or more, any eleven or more, any twelve, any thirteen or more, any fourteen or more, any fifteen or more, any sixteen or more, any seventeen or more, any eighteen or more, any nineteen or more, any twenty or more, any twenty one or more, any twenty two or more, any twenty three or more, any twenty four or more, any twenty five or more, any twenty-six or more, any twenty-seven or more, any twenty-eight or more, any twenty-nine or more, any thirty or more, any thirty-one or more, any thirty-two or more, any thirty-three or more, any thirty-four or more, any thirty-five or more, any thirty-six or more, any thirty-seven or more, any thirty-eight or more, any thirty-nine or more, any forty or more, any forty-one or more, any forty-two or more, any forty-three or more, any forty-four or more, any forty-five or more, any forty-six or more, any forty-seven or more, any forty-eight or more, or all forth-nine of ADA, ADAMTS2, AKAP2, ARHGEF25, ARRB1, ARRDC2, ATOH8, CLEC4C, CPSF7, CUX2, FKBP5, FSTL3, GSTA3, HEG1, IGIP, INO80C, JAG1, JUN, KRT5, LEP, LILRA4, MRPS35, MSMP, NES, NFE2L1, NR4A2, NTRK2, PACSIN1, PER1, PLD4, PLEK, PRG2, RAP1GAP2, RGP1, SEMA3G, SH3PXD2A, SKIL, SMPD3, SPEG, SRPX, SYNPO, TEAD4, TIPARP, TNFRSF21, TPST1, TRPS1, UBE2Q1, VSIG4, and ZNF768; or
(j) any one or more, any two or more, any three or more, any four or more, any five or more, any six or more, any seven or more, any eight or more, any nine or more, any ten or more, any eleven or more, any twelve or more, or all thirteen of AKAP2, ARRB1, CPSF7, INO80C, JAG1, MSMP, NR4A2, PLEK, RAP1GAP2, SPEG, TRPS1, UBE2Q1, and ZNF768.

In some aspects of a method of the present invention, the method further includes performing prenatal genetic screening testing or prenatal genetic diagnostic testing on a portion of the biosample obtained from a pregnant female.

The present invention includes a circulating RNA (C-RNA) signature for preeclampsia or for an elevated risk of preeclampsia, the C-RNA signature comprising any one or more, any two or more, any three or more, or all four of HCG4P8, EYS, AGGF1P10, and GLYATL2.

The present invention includes a solid support array comprising a plurality of agents capable of binding and/or identifying a C-RNA signature comprising any one or more, any two or more, any three or more, or all four of HCG4P8, EYS, AGGF1P10, and GLYATL2.

The present invention includes a kit comprising a plurality of probes capable of binding and/or identifying a C-RNA signature comprising any one or more, any two or more, any three or more, or all four of HCG4P8, EYS, AGGF1P10, and GLYATL2.

The present invention includes a kit comprising a plurality of primers for selectively amplifying a C-RNA signature comprising any one or more, any two or more, any three or more, or all four of HCG4P8, EYS, AGGF1P10, and GLYATL2.

As used herein, the term “nucleic acid” is intended to be consistent with its use in the art and includes naturally occurring nucleic acids or functional analogs thereof. Particularly useful functional analogs are capable of hybridizing to a nucleic acid in a sequence specific fashion or capable of being used as a template for replication of a particular nucleotide sequence. Naturally occurring nucleic acids generally have a backbone containing phosphodiester bonds. An analog structure can have an alternate backbone linkage including any of a variety of those known in the art. Naturally occurring nucleic acids generally have a deoxyribose sugar (for example, found in deoxyribonucleic acid (DNA)) or a ribose sugar (for example, found in ribonucleic acid (RNA)). A nucleic acid can contain any of a variety of analogs of these sugar moieties that are known in the art. A nucleic acid can include native or non-native bases. In this regard, a native deoxyribonucleic acid can have one or more bases selected from the group consisting of adenine, thymine, cytosine or guanine and a ribonucleic acid can have one or more bases selected from the group consisting of uracil, adenine, cytosine, or guanine. Useful non-native bases that can be included in a nucleic acid are known in the art. The term “template” and “target,” when used in reference to a nucleic acid, is intended as a semantic identifier for the nucleic acid in the context of a method or composition set forth herein and does not necessarily limit the structure or function of the nucleic acid beyond what is otherwise explicitly indicated.

As used herein, “amplify,” “amplifying” or “amplification reaction” and their derivatives, refer generally to any action or process whereby at least a portion of a nucleic acid molecule is replicated or copied into at least one additional nucleic acid molecule. The additional nucleic acid molecule optionally includes sequence that is substantially identical or substantially complementary to at least some portion of the target nucleic acid molecule. The target nucleic acid molecule can be single-stranded or double-stranded and the additional nucleic acid molecule can independently be single-stranded or double-stranded. Amplification optionally includes linear or exponential replication of a nucleic acid molecule. In some embodiments, such amplification can be performed using isothermal conditions; in other embodiments, such amplification can include thermocycling. In some embodiments, the amplification is a multiplex amplification that includes the simultaneous amplification of a plurality of target sequences in a single amplification reaction. In some embodiments, “amplification” includes amplification of at least some portion of DNA and RNA based nucleic acids alone, or in combination. The amplification reaction can include any of the amplification processes known to one of ordinary skill in the art. In some embodiments, the amplification reaction includes polymerase chain reaction (PCR).

As used herein, “amplification conditions” and its derivatives, generally refers to conditions suitable for amplifying one or more nucleic acid sequences. Such amplification can be linear or exponential. In some embodiments, the amplification conditions can include isothermal conditions or alternatively can include thermocyling conditions, or a combination of isothermal and thermocycling conditions. In some embodiments, the conditions suitable for amplifying one or more nucleic acid sequences include polymerase chain reaction (PCR) conditions. Typically, the amplification conditions refer to a reaction mixture that is sufficient to amplify nucleic acids such as one or more target sequences, or to amplify an amplified target sequence ligated to one or more adapters, e.g., an adapter-ligated amplified target sequence. Generally, the amplification conditions include a catalyst for amplification or for nucleic acid synthesis, for example a polymerase; a primer that possesses some degree of complementarity to the nucleic acid to be amplified; and nucleotides, such as deoxyribonucleotide triphosphates (dNTPs) to promote extension of the primer once hybridized to the nucleic acid. The amplification conditions can require hybridization or annealing of a primer to a nucleic acid, extension of the primer and a denaturing step in which the extended primer is separated from the nucleic acid sequence undergoing amplification. Typically, but not necessarily, amplification conditions can include thermocycling; in some embodiments, amplification conditions include a plurality of cycles where the steps of annealing, extending, and separating are repeated. Typically, the amplification conditions include cations such as Mg⁺⁺ or Mn⁺⁺ and can also include various modifiers of ionic strength.

As used herein, the term “polymerase chain reaction” (PCR) refers to the method of U.S. Pat. Nos. 4,683,195 and 4,683,202, which describes a method for increasing the concentration of a segment of a polynucleotide of interest in a mixture of genomic DNA without cloning or purification. This process for amplifying the polynucleotide of interest consists of introducing a large excess of two oligonucleotide primers to the DNA mixture containing the desired polynucleotide of interest, followed by a series of thermal cycling in the presence of a DNA polymerase. The two primers are complementary to their respective strands of the double-stranded polynucleotide of interest. The mixture is denatured at a higher temperature first and the primers are then annealed to complementary sequences within the polynucleotide of interest molecule. Following annealing, the primers are extended with a polymerase to form a new pair of complementary strands. The steps of denaturation, primer annealing and polymerase extension can be repeated many times (referred to as thermocycling) to obtain a high concentration of an amplified segment of the desired polynucleotide of interest. The length of the amplified segment of the desired polynucleotide of interest (amplicon) is determined by the relative positions of the primers with respect to each other, and therefore, this length is a controllable parameter. By virtue of repeating the process, the method is referred to as the “polymerase chain reaction” (hereinafter “PCR”). Because the desired amplified segments of the polynucleotide of interest become the predominant nucleic acid sequences (in terms of concentration) in the mixture, they are said to be “PCR amplified.” In a modification to the method discussed above, the target nucleic acid molecules can be PCR amplified using a plurality of different primer pairs, in some cases, one or more primer pairs per target nucleic acid molecule of interest, thereby forming a multiplex PCR reaction.

As used herein, the term “primer” and its derivatives refer generally to any polynucleotide that can hybridize to a target sequence of interest. Typically, the primer functions as a substrate onto which nucleotides can be polymerized by a polymerase; in some embodiments, however, the primer can become incorporated into the synthesized nucleic acid strand and provide a site to which another primer can hybridize to prime synthesis of a new strand that is complementary to the synthesized nucleic acid molecule. The primer can include any combination of nucleotides or analogs thereof. In some embodiments, the primer is a single-stranded oligonucleotide or polynucleotide. The terms “polynucleotide” and “oligonucleotide” are used interchangeably herein to refer to a polymeric form of nucleotides of any length, and may comprise ribonucleotides, deoxyribonucleotides, analogs thereof, or mixtures thereof. The terms should be understood to include, as equivalents, analogs of either DNA or RNA made from nucleotide analogs and to be applicable to single stranded (such as sense or antisense) and double-stranded polynucleotides. The term as used herein also encompasses cDNA, that is complementary or copy DNA produced from an RNA template, for example by the action of reverse transcriptase. This term refers only to the primary structure of the molecule. Thus, the term includes triple-, double- and single-stranded deoxyribonucleic acid (“DNA”), as well as triple-, double- and single-stranded ribonucleic acid (“RNA”).

As used herein, the terms “library” and “sequencing library” refer to a collection or plurality of template molecules which share common sequences at their 5′ ends and common sequences at their 3′ ends. The collection of template molecules containing known common sequences at their 3′ and 5′ ends may also be referred to as a 3′ and 5′ modified library.

The term “flowcell” as used herein refers to a chamber comprising a solid surface across which one or more fluid reagents can be flowed. Examples of flowcells and related fluidic systems and detection platforms that can be readily used in the methods of the present disclosure are described, for example, in Bentley et al., Nature 456:53-59 (2008), WO 04/018497; U.S. Pat. No. 7,057,026; WO 91/06678; WO 07/123744; U.S. Pat. Nos. 7,329,492; 7,211,414; 7,315,019; 7,405,281, and US 2008/0108082.

As used herein, the term “array” refers to a population of sites that can be differentiated from each other according to relative location. Different molecules that are at different sites of an array can be differentiated from each other according to the locations of the sites in the array. An individual site of an array can include one or more molecules of a particular type. For example, a site can include a single target nucleic acid molecule having a particular sequence or a site can include several nucleic acid molecules having the same sequence (and/or complementary sequence, thereof). The sites of an array can be different features located on the same substrate. Exemplary features include without limitation, wells in a substrate, beads (or other particles) in or on a substrate, projections from a substrate, ridges on a substrate or channels in a substrate. The sites of an array can be separate substrates each bearing a different molecule. Different molecules attached to separate substrates can be identified according to the locations of the substrates on a surface to which the substrates are associated or according to the locations of the substrates in a liquid or gel. Exemplary arrays in which separate substrates are located on a surface include, without limitation, those having beads in wells.

The term “Next Generation Sequencing (NGS)” herein refers to sequencing methods that allow for massively parallel sequencing of clonally amplified molecules and of single nucleic acid molecules. Non-limiting examples of NGS include sequencing-by-synthesis using reversible dye terminators, and sequencing-by-ligation.

The term “sensitivity” as used herein is equal to the number of true positives divided by the sum of true positives and false negatives.

The term “specificity” as used herein is equal to the number of true negatives divided by the sum of true negatives and false positives.

The term “enrich” herein refers to the process of amplifying nucleic acids contained in a portion of a sample. Enrichment includes specific enrichment that targets specific sequences, e.g., polymorphic sequences, and non-specific enrichment that amplifies the whole genome of the DNA fragments of the sample.

As used herein, the term “each,” when used in reference to a collection of items, is intended to identify an individual item in the collection but does not necessarily refer to every item in the collection unless the context clearly dictates otherwise.

As used herein, “providing” in the context of a composition, an article, a nucleic acid, or a nucleus means making the composition, article, nucleic acid, or nucleus, purchasing the composition, article, nucleic acid, or nucleus, or otherwise obtaining the compound, composition, article, or nucleus.

The term “and/or” means one or all of the listed elements or a combination of any two or more of the listed elements. The words “preferred” and “preferably” refer to embodiments of the disclosure that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the disclosure.

The terms “comprises” and variations thereof do not have a limiting meaning where these terms appear in the description and claims.

It is understood that wherever embodiments are described herein with the language “include,” “includes,” or “including,” and the like, otherwise analogous embodiments described in terms of “consisting of” and/or “consisting essentially of” are also provided.

Unless otherwise specified, “a,” “an,” “the,” and “at least one” are used interchangeably and mean one or more than one.

Also herein, the recitations of numerical ranges by endpoints include all numbers subsumed within that range (e.g., 1 to 5 includes 1, 1.5, 2, 2.75, 3, 3.80, 4, 5, etc.).

Reference throughout this specification to “one embodiment,” “an embodiment,” “certain embodiments,” or “some embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more embodiments.

For any method disclosed herein that includes discrete steps, the steps may be conducted in any feasible order. And, as appropriate, any combination of two or more steps may be conducted simultaneously.

The above summary of the present disclosure is not intended to describe each disclosed embodiment or every implementation of the present disclosure. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1. Overview of Pregnancy outcome prediction study.

FIGS. 2A and 2B. DEX transcript data. Graphs plot GA (x-axis) versus transcript abundance (y-axis, log 2(CPM)). With the top row each line is data from one patient. With the bottom row, each line is mean+standard deviation of data from all patients within a group. Orange=preeclampsia (PE) patients. Gray=control patients. FIG. 2A presents data for HCG4P8 and EYS. FIG. 2B presents data for AGGF1P10 and GLYATL2.

The schematic drawings are not necessarily to scale. Like numbers used in the figures may refer to like components. However, it will be understood that the use of a number to refer to a component in a given figure is not intended to limit the component in another figure labeled with the same number. In addition, the use of different numbers to refer to components is not intended to indicate that the different numbered components cannot be the same or similar to other numbered components.

### DETAILED DESCRIPTION

Provided herein are circulating RNAs found in the maternal circulation, the abundance of which are altered in subjects prior to a diagnosis of preeclampsia and the use of these circulating RNA transcripts in noninvasive methods for the diagnosis of preeclampsia and the identification of pregnant women at risk for developing preeclampsia. These circulating RNAs include HCG4P8, EYS, AGGF1P10, and GLYATL2. These C-RNA transcripts function as biomarkers of fetal, placental, and/or maternal health, and are useful in the identification of pregnant women at risk for developing preeclampsia, including, but not limited to early onset preeclampsia, with onset before 34 weeks of gestation, and/or late onset preeclampsia, with onset at 34 weeks or more of gestation.

While most of the DNA and RNA in the body is located within cells, extracellular nucleic acids can also be found circulating freely in the blood. Circulating RNA, also referred to herein as “C-RNA,” refers to extracellular segments of RNA found in the bloodstream. C-RNA molecules originate predominately from two sources: one, released into the circulation from dying cells undergoing apoptosis, and two, contained within exosomes shed by living cells into the circulation. Exosomes are small membranous vesicles about 30-150 nm of diameter released from many cell types into the extracellular space and are found in a wide variety of body fluids, including serum, urine, and breast milk, and carrying protein, mRNA, and microRNA.

As circulating RNA is released by all tissues into the bloodstream, it offers an accessible measurement of placental, fetal, and maternal health (Koh et al., 2014, Proceedings of the National Academy of Sciences; 111:7361-7366; and Tsui et al., 2014, Clinical Chemistry; 60:954-962). Several studies have begun to investigate and identify potential biomarkers in C-RNA for a range of pregnancy complications, including preterm birth, PE, and infectious disease (Pan et al., 2017, Clinical Chemistry; 63:1695-1704; Whitehead et al., 2016, Prenatal Diagnosis; 36:997-1008; Tsang et al., 2017, Proc Natl Acad Sci USA; 114: E7786-E7795; and Ngo et al., 2018, Science; 360:1133-1136). However, these studies have involved few patients and have been limited to monitoring small numbers of genes—almost exclusively placental and fetal derived transcripts. Further, the significant interindividual variability in this sample type threatens to obscure subtle changes in disease specific biomarkers (Meder et al. 2014, Clin Chem; 60:1200-1208). Measurements of the entire circulating transcriptome are difficult to perform because they require specific upfront sample collection and processing to minimize variability and contamination from cell lysis (Chiu et al., 2001, Clinical Chemistry; 47:1607-1613; and Page et al., 2013, PLoS ONE; 8: e77963). This complex workflow makes large clinical sample collections difficult to achieve because the labor required for immediate processing of blood samples is infeasible for many clinics (Marton and Weiner, 2013, BioMed Research International; 2013:891391). Therefore, many PE-focused C-RNA studies have measured RNA of previously identified serum protein biomarkers, including soluble FLT1, soluble endoglin, and oxidative stress and angiogenic markers (Nakamura et al., 2009, Prenat Diagn; 29:691-696; Purwosunu et al., 2009, Reprod Sci; 16:857-864; and Paiva et al., 2011, J Clin Endocrinol Metab; 96:E1807-1815). While protein measurements of these serum markers are known to be altered in PE (Maynard et al., 2003, J Clin Invest; 111:649-658; Venkatesha et al., 2006, Nat Med; 12:642-649; and Rana et al., 2018, Pregnancy Hypertens; 13:100-106), it is unknown whether they will serve as the most effective predictors in C-RNA, thus warranting a broader discovery approach.

Preeclampsia (PE) is one of the most common and serious complications of pregnancy, affecting an estimated 4-5% of pregnancies worldwide (Abalos et al., 2013, Eur J Obstet Gynecol Reprod Biol; 170:1-7; and Ananth et al., 2013, BMJ; 347:f6564) and is associated with substantial maternal and perinatal morbidity and mortality (Kuklina et al., 2009, Obstet Gynecol; 113:1299; and Basso et al., 2006, JAMA; 296:1357-1362). In the United States, the incidence of PE is increasing due to advanced maternal age and the increasing prevalence of comorbid conditions such as obesity (Spradley et al., 2015, Biomolecules; 5:3142-3176), costing the US healthcare system an estimated 2 billion dollars annually (Stevens et al., 2017, Am J Obstet Gynecol; 217:237-248.e16). PE is a heterogeneous disorder and associated with different severity and patient outcomes based on whether it manifests before (early-onset) or after (late-onset) 34 gestational weeks (Staff et al., 2013, Hypertension; 61:932-942; Chaiworapongsa et al., 2014, Nature Reviews Nephrology; 10, 466-4803; and Dadelszen et al., 2003, Hypertension in Pregnancy; 22:143-148).

Preeclampsia is placental in origin but gains a substantial maternal component as the disease progresses (Staff et al., 2013, Hypertension; 61:932-942; and Chaiworapongsa et al., 2014, Nature Reviews Nephrology; 10, 466-480). Yet, to date, purported biomarkers have shown limited clinical utility (Poon and Nicolaides, 2014, Obstetrics and Gynecology International; 2014:1-11; Zeisler et al., 2016, N Engl J Med; 374:13-22; and Duhig et al., 2018, F1000Research; 7:242). The pathophysiology of early-onset PE is incompletely understood, but is thought to occur in two phases (Phipps et al., 2019, Nature Reviews Nephrology; 15:275). Early-onset PE originates with abnormal implantation and placentation in the first trimester, related to maternal immune dysfunction (Hiby et al., 2010, J Clin Invest; 120:4102-4110; Ratsep et al., 2015, Reproduction; 149:R91-R102; and Girardi, 2018, Semin Immunopathol; 40:103-111), incomplete cytotrophoblast differentiation (Zhou et al., 1997, J Clin Invest; 99:2152-2164), and/or oxidative stress at the maternal-placental interface (Burton and Jauniaux, 2011, Best Pract Res Clin Obstet Gynaecol; 25:287-299), resulting in incomplete remodeling of the maternal spiral arteries and failure to establish the definitive uteroplacental circulation (Lyall et al., 2013, Hypertension; 62:1046-1054). This leads to inadequate placental perfusion after 20 weeks' gestation. The resultant placental dysregulation triggers phase two, which manifests predominantly as maternal systemic vascular dysfunction with negative consequences for the fetus, including fetal growth restriction and iatrogenic preterm birth (Hecht et al., 2017, Hypertens Pregnancy; 36:259-268; Young et al., 2010, Annu Rev Pathol; 5:173-192; and Backes et al., 2011, J Pregnancy; 2011:doi:10.1155/2011/214365. In contrast, the placental dysfunction in late-onset PE is thought to be due not to abnormal placentation, but to disturbance in placental perfusion resulting from maternal vascular disease, such as that seen in patients with chronic hypertension, pregestational diabetes (Vambergue and Fajardy, 2011, World J Diabetes; 2:196-203), and collagen vascular disorders (“Placental pathology in maternal autoimmune diseases-new insights and clinical implications,” 2017, International Journal of Reproduction, Contraception, Obstetrics and Gynecology; 6:4090-4097).

The heterogeneity and complexity of PE have made it difficult to diagnose, to predict risk, and to develop treatments. Further, the inability to easily interrogate the primary affected organ, the placenta, has limited molecular characterization of disease progression. Thus, the biomarkers provided herein, HCG4P8, EYS, AGGF1P10, and GLYATL2, associated with preeclampsia, are useful as predictive, prognostic, and diagnostic biomarkers of the disease.

As described in the examples section included herewith and using a workflow similar to that described in WO 2019/227015, WO 2021/102236, and Munchel et al., 2020, Sci Transl Med; 12(550):eaaz0131. doi: 10.1126/scitranslmed.aaz0131 (all of which are hereby incorporated by reference), C-RNAs that serve as biomarkers for the diagnosis and prediction of at-risk pregnancies were identified. Specifically, transcripts for HCG4P8, EYS, AGGF1P10, and GLYATL2 were found to be altered in the circulating RNA of pregnant women prior to a diagnosis of preeclampsia. HCG4P8, also known as HLA Complex Group 4 Pseudogene 8 (HGNC: 22927; NCBI Entrez Gene: 353005; Ensembl: ENSG00000229142), is an HLA complex pseudogene. EYS, also known as Eyes Shut Homolog (HGNC: 215555; ENSG00000188107), is an epidermal growth factor-like protein that maintains photoreceptor cells and plays a role in protein trafficking. AGGF1P10, also known as Angiogenic Factor With G-Patch And FHA Domains 1 Pseudogene 10 (HGNC: 51747; NCBI Entrez Gene: 100288774; Ensembl: ENSG00000282968), is an angiogenesis factor pseudogene. GLYATL2, also known as Glycine-N-Acyltransferase Like 2 (HGNC: 24178; NCBI Entrez Gene 219970; Ensembl: ENSG00000156689; OMIM®: 614762; UniProtKB/Swiss-Prot: Q8WU03), enables glycine N-acyltransferase activity and is involved in lipid catabolism and signaling. The identification and/or quantification of one or more of these C-RNA biomarkers within a sample obtained from a subject can be used to determine that the subject suffers from preeclampsia or is at a risk of developing preeclampsia.

In some aspects, the abundance of C-RNA encoding HCG4P8, EYS, and/or AGGF1P10 is down regulated. As used herein, down regulated indicates that the prevalence of the C-RNA biomarker in the sample is decreased in abundance in comparison to an appropriate control. A subject may be diagnosed with preeclampsia or determined to be at risk for developing preeclampsia based on a statistically significant (p<0.05) decreases in the abundance of C-RNA encoding HCG4P8, EYS, and/or AGGF1P10 relative to the corresponding data of an appropriate control sample.

In some aspects, the abundance of C-RNA encoding GLYATL2 is up regulated. As used herein, up regulated indicates that the prevalence of the C-RNA biomarker in the sample is increased in comparison to an appropriate control. A subject may be diagnosed with preeclampsia or determined to be at risk for developing preeclampsia based on a statistically significant (p<0.05) increase of the abundance of C-RNA encoding GLYATL2 relative to the corresponding data of an appropriate control sample.

An appropriate control may be a normal control, for example, a sample from a healthy, gestational age-matched pregnant female. In some embodiments, the results of an appropriate control have been previously obtained and are recorded and stored as historical results, on for example, an electronic storage medium.

In some embodiments, a determination of C-RNA abundance of only one of HCG4P8, EYS, AGGF1P10, or GLYATL2 is determined for a diagnosis of preeclampsia or a determination of a risk for developing preeclampsia.

In some embodiments, a determination of C-RNA abundance of any two of HCG4P8, EYS, AGGF1P10, or GLYATL2 (for example, HCG4P8 and EYS, HCG4P8 and AGGF1P10, HCG4P8 and GLYATL2, EYS and AGGF1P10, EYS and GLYATL2, or AGGF1P10 and GLYATL2) is determined for a diagnosis of preeclampsia or a determination of a risk for developing preeclampsia.

In some embodiments, a determination of C-RNA abundance of any three of HCG4P8, EYS, AGGF1P10, or GLYATL2 (for example, HCG4P8, EYS, and AGGF1P10; HCG4P8, EYS, and GLYATL2; HCG4P8, AGGF1P10, and GLYATL2; or EYS, AGGF1P10, and GLYATL2) is determined for a diagnosis of preeclampsia or a determination of a risk for developing preeclampsia.

In some embodiments, a determination of C-RNA abundance of all four of HCG4P8, EYS, AGGF1P10, and GLYATL2 is determined for a diagnosis of preeclampsia or a determination of a risk for developing preeclampsia.

In some embodiments, a C-RNA signature within maternal circulation indicative of preeclampsia includes C-RNA molecules encoding at least a portion of a protein selected from HCG4P8, EYS, AGGF1P10, and/or GLYATL2. With encoding at least a portion of a protein, also referred to herein as encoding a coding region of a protein, a C-RNA molecule encodes a sufficient number of amino acids to enable the listed protein(s) to be identified as such.

Such a C-RNA signature within the maternal circulation may further include one or more C-RNA molecules encoding at least a portion of a protein selected from any one or more, any two or more, any three or more, any four or more, any five or more, any six or more, any seven or more, any eight or more, any nine or more, any ten or more, any eleven or more, any twelve, any thirteen or more, any fourteen or more, any fifteen or more, any sixteen or more, any seventeen or more, any eighteen or more, any nineteen or more, any twenty or more, any twenty one or more, any twenty two or more, any twenty three or more, any twenty four or more, any twenty five or more, any fifty or more, any seventy or more, or all seventy-five of ARRDC2, JUN, SKIL, ATP13A3, PDE8B, GSTA3, PAPPA2, TIPARP, LEP, RGP1, USP54, CLEC4C, MRPS35, ARHGEF25, CUX2, HEATR9, FSTL3, DDI2, ZMYM6, ST6GALNAC3, GBP2, NES, ETV3, ADAM17, ATOH8, SLC4A3, TRAF3IP1, TTC21A, HEG1, ASTE1, TMEM108, ENC1, SCAMPI, ARRDC3, SLC26A2, SLIT3, CLIC5, TNFRSF21, PPP1R17, TPST1, GATSL2, SPDYE5, HIPK2, MTRNR2L6, CLCN1, GINS4, CRH, C10orf2, TRUB1, PRG2, ACY3, FAR2, CD63, CKAP4, TPCN1, RNF6, THTPA, FOS, PARN, ORAI3, ELMO3, SMPD3, SERPINF1, TMEM11, PSMD11, EBI3, CLEC4M, CCDC151, CPAMD8, CNFN, LILRA4, ADA, C22orf39, PI4KAP1, and ARFGAP3. This C-RNA signature is the Adaboost General signature obtained with the TruSeq library prep method identified as list (a), as described in more detail in WO 2019/227015 and WO 2021/102236.

Such a C-RNA signature within the maternal circulation may further include one or more C-RNA molecules encoding at least a portion of a protein selected from any one or more, any two or more, any three or more, any four or more, any five or more, any six or more, any seven or more, any eight or more, any nine or more, any ten or more, any eleven or more, any twelve or more, any thirteen or more, any fourteen or more, any fifteen or more, any sixteen or more, any seventeen or more, any eighteen or more, any nineteen or more, any twenty or more, any twenty one or more, any twenty two or more, any twenty three or more, any twenty four or more, any twenty five or more, any twenty six of more, or all twenty-seven of TIMP4, FLG, HTRA4, AMPH, LCN6, CRH, TEAD4, ARMS2, PAPPA2, SEMA3G, ADAMTS1, ALOX15B, SLC9A3R2, TIMP3, IGFBP5, HSPA12B, CLEC4C, KRT5, PRG2, PRX, ARHGEF25, ADAMTS2, DAAM2, FAM107A, LEP, NES, and VSIG4. This C-RNA signature is the Bootstrapping signature obtained with the TruSeq library prep method identified as list (b), as described in more detail in WO 2019/227015 and WO 2021/102236.

Such a C-RNA signature within the maternal circulation may further include one or more C-RNA molecules encoding at least a portion of a protein selected from any one or more, any two or more, any three or more, any four or more, any five or more, any six or more, any seven or more, any eight or more, any nine or more, any ten or more, any eleven or more, any twelve, any thirteen or more, any fourteen or more, any fifteen or more, any sixteen or more, any seventeen or more, any eighteen or more, any nineteen or more, any twenty or more, any twenty one or more, any twenty two or more, any twenty three or more, any twenty four or more, any twenty five or more, any fifty or more, any seventy-five or more, any one hundred or more, or all one hundred twenty-two of CYP26B1, IRF6, MYH14, PODXL, PPP1R3C, SH3RF2, TMC7, ZNF366, ADCY1, C6, FAM219A, HAO2, IGIP, IL1R2, NTRK2, SH3PXD2A, SSUH2, SULT2A1, FMO3, FSTL3, GATA5, HTRA1, C8B, H19, MN1, NFE2L1, PRDM16, AP3B2, EMP1, FLNC, STAG3, CPB2, TENC1, RP1L1, A1CF, NPR1, TEK, ERRFIl, ARHGEF15, CD34, RSPO3, ALPK3, SAMD4A, ZCCHC24, LEAP2, MYL2, NRG3, ZBTB16, SERPINA3, AQP7, SRPX, UACA, ANO1, FKBP5, SCN5A, PTPN21, CACNAlC, ERG, SOX17, WWTR1, AIF1L, CA3, HRG, TAT, AQP7P1, ADRA2C, SYNPO, FN1, GPR116, KRT17, AZGP1, BCL6B, KIF1C, CLIC5, GPR4, GJA5, OLAH, C14orf37, ZEB1, JAG2, KIF26A, APOLD1, PNMT, MYOM3, PITPNM3, TIMP4, HTRA4, AMPH, LCN6, CRH, TEAD4, ARMS2, PAPPA2, SEMA3G, ADAMTS1, ALOX15B, SLC9A3R2, TIMP3, IGFBP5, HSPA12B, PRG2, PRX, ARHGEF25, ADAMTS2, DAAM2, FAM107A, LEP, NES, VSIG4, HBG2, CADM2, LAMP5, PTGDR2, NOMO1, NXF3, PLD4, BPIFB3, PACSIN1, CUX2, FLG, CLEC4C, and KRT5. This C-RNA signature is the Standard DEX Treat signature obtained with the TruSeq library prep method identified as list (c) and as described in more detail in WO 2019/227015 and WO 2021/102236.

Such a C-RNA signature within the maternal circulation may further include one or more C-RNA molecules encoding at least a portion of a protein selected from any one or more, any two or more, any three or more, any four or more, any five or more, any six or more, any seven or more, any eight or more, any nine or more, any ten or more, any eleven or more, any twelve or more, any thirteen or more, any fourteen or more, any fifteen or more, any sixteen or more, any seventeen or more, any eighteen or more, any nineteen or more, any twenty or more, any twenty-one or more, any twenty-two or more, any twenty-three or more, any twenty-four or more, any twenty-five or more, any twenty-six or more, any twenty-seven or more, any twenty-eight or more, any twenty-nine or more, or all thirty of VSIG4, ADAMTS2, NES, FAM107A, LEP, DAAM2, ARHGEF25, TIMP3, PRX, ALOX15B, HSPA12B, IGFBP5, CLEC4C, SLC9A3R2, ADAMTS1, SEMA3G, KRT5, AMPH, PRG2, PAPPA2, TEAD4, CRH, PITPNM3, TIMP4, PNMT, ZEB1, APOLD1, PLD4, CUX2, and HTRA4. This C-RNA signature is the Jacknifing signature obtained with the TruSeq library prep method identified as list (d), as described in more detail in WO 2019/227015 and WO 2021/102236.

Such a C-RNA signature within the maternal circulation may further include one or more C-RNA molecules encoding at least a portion of a protein selected from any one or more, any two or more, any three or more, any four or more, any five or more, any six or more, any seven or more, any eight or more, any nine or more, any ten or more, any eleven or more, any twelve or more, any thirteen or more, any fourteen or more, any fifteen or more, any sixteen or more, any seventeen or more, any eighteen or more, any nineteen or more, any twenty or more, any twenty-one or more, any twenty-two or more, any twenty-three or more, any twenty-four or more, any twenty-five or more, or all twenty-six of ADAMTS1, ADAMTS2, ALOX15B, AMPH, ARHGEF25, CELF4, DAAM2, FAM107A, HSPA12B, HTRA4, IGFBP5, KCNA5, KRT5, LCN6, LEP, LRRC26, NES, OLAH, PACSIN1, PAPPA2, PRX, PTGDR2, SEMA3G, SLC9A3R2, TIMP3, and VSIG4. This C-RNA signature is the Standard DEX Treat signature obtained with the Nextera Flex for Enrichment library prep method identified as list (e), as described in more detail in WO 2019/227015 and WO 2021/102236.

Such a C-RNA signature within the maternal circulation may further include one or more C-RNA molecules encoding at least a portion of a protein selected from any one or more, any two or more, any three or more, any four or more, any five or more, any six or more, any seven or more, any eight or more, any nine or more, any ten or more, any eleven or more, any twelve or more, any thirteen or more, any fourteen or more, any fifteen or more, any sixteen or more, any seventeen or more, any eighteen or more, any nineteen or more, any twenty or more, any twenty-one or more, or all twenty-two of ADAMTS1, ADAMTS2, ALOX15B, ARHGEF25, CELF4, DAAM2, FAM107A, HTRA4, IGFBP5, KCNA5, KRT5, LCN6, LEP, LRRC26, NES, OLAH, PRX, PTGDR2, SEMA3G, SLC9A3R2, TIMP3, and VSIG4. This C-RNA signature is the Jacknifing signature obtained with the Nextera Flex for Enrichment library method identified as list (f) and as described in more detail in WO 2019/227015 and WO 2021/102236.

Such a C-RNA signature within the maternal circulation may further include one or more C-RNA molecules encoding at least a portion of a protein selected from any one or more, any two or more, any three or more, any four or more, any five or more, any six or more, any seven or more, any eight or more, any nine or more, any ten or more, or all eleven of CLEC4C, ARHGEF25, ADAMTS2, LEP, ARRDC2, SKIL, PAPPA2, VSIG4, ARRDC4, CRH, and NES. This C-RNA signature is the Adaboost Refined TruSeq signature obtained with the TruSeq library prep method identified as list (g), as described in more detail in WO 2019/227015 and WO 2021/102236.

Such a C-RNA signature within the maternal circulation may further include one or more C-RNA molecules encoding at least a portion of a protein selected from ADAMTS2, ARHGEF25, ARRDC2, CLEC4C, LEP, PAPPA2, and VSIG4 (also referred to as “AdaBoost Refined 2,” as described in more detail in WO 2019/227015 and WO 2021/102236), ADAMTS2, ARHGEF25, ARRDC2, CLEC4C, LEP, PAPPA2, SKIL, and VSIG4 (also referred to as “AdaBoost Refined 3,” as described in more detail in WO 2019/227015 and WO 2021/102236), ADAMTS2, ARHGEF25, ARRDC4, CLEC4C, LEP, NES, SKIL, and VSIG4 (also referred to as “AdaBoost Refined 4,” as described in more detail in WO 2019/227015 and WO 2021/102236), ADAMTS2, ARHGEF25, ARRDC2, ARRDC4, CLEC4C, CRH, LEP, PAPPA2, SKIL, and VSIG4 (also referred to as “AdaBoost Refined 5,” as described in more detail in WO 2019/227015 and WO 2021/102236), ADAMTS2, ARHGEF25, ARRDC2, CLEC4C, LEP, and SKIL (also referred to as “AdaBoost Refined 6,” as described in more detail in WO 2019/227015 and WO 2021/102236), or ADAMTS2, ARHGEF25, ARRDC2, ARRDC4, CLEC4C, LEP, PAPPA2, and SKIL (also referred to as “AdaBoost Refined 7,” as described in more detail in WO 2019/227015 and WO 2021/102236).

Such a C-RNA signature within the maternal circulation may further include one or more C-RNA molecules encoding at least a portion of a protein selected from any one or more, two or more, any three or more, any four or more, any five or more, any six or more, any seven or more, any eight or more, any nine or more, any ten or more, any eleven or more, any twelve or more, any thirteen or more, any fourteen or more, any fifteen or more, any sixteen or more, any seventeen or more, any eighteen or more, any nineteen or more, any twenty or more, any twenty-one or more, any twenty-two or more, any twenty-three or more, or all twenty-four of LEP, PAPPA2, KCNA5, ADAMTS2, MYOM3, ATP13A3, ARHGEF25, ADA, HTRA4, NES, CRH, ACY3, PLD4, SCT, NOX4, PACSIN1, SERPINF1, SKIL, SEMA3G, TIPARP, LRRC26, PHEX, LILRA4, and PER1. This C-RNA signature is the Adaboost Refined Nextera Flex signature obtained with the Nextera Flex for Enrichment library prep method identified as list (h), as described in more detail in WO 2019/227015 and WO 2021/102236.

Such a C-RNA signature within the maternal circulation may further include one or more C-RNA molecules encoding at least a portion of a protein selected from any one or more, any two or more, any three or more, any four or more, any five or more, any six or more, any seven or more, any eight or more, any nine or more, any ten or more, any eleven or more, any twelve, any thirteen or more, any fourteen or more, any fifteen or more, any sixteen or more, any seventeen or more, any eighteen or more, any nineteen or more, any twenty or more, any twenty one or more, any twenty two or more, any twenty three or more, any twenty four or more, any twenty five or more, any twenty-six or more, any twenty-seven or more, any twenty-eight or more, any twenty-nine or more, any thirty or more, any thirty-one or more, any thirty-two or more, any thirty-three or more, any thirty-four or more, any thirty-five or more, any thirty-six or more, any thirty-seven or more, any thirty-eight or more, any thirty-nine or more, any forty or more, any forty-one or more, any forty-two or more, any forty-three or more, any forty-four or more, any forty-five or more, any forty-six or more, any forty-seven or more, any forty-eight or more, or all forth-nine of ADA, ADAMTS2, AKAP2, ARHGEF25, ARRB1, ARRDC2, ATOH8, CLEC4C, CPSF7, CUX2, FKBP5, FSTL3, GSTA3, HEG1, IGIP, INO80C, JAG1, JUN, KRT5, LEP, LILRA4, MRPS35, MSMP, NES, NFE2L1, NR4A2, NTRK2, PACSIN1, PER1, PLD4, PLEK, PRG2, RAP1GAP2, RGP1, SEMA3G, SH3PXD2A, SKIL, SMPD3, SPEG, SRPX, SYNPO, TEAD4, TIPARP, TNFRSF21, TPST1, TRPS1, UBE2Q1, VSIG4, and ZNF768. This C-RNA signature is identified as list (i), as described in more detail in WO 2021/102236.

Such a C-RNA signature within the maternal circulation may further include one or more C-RNA molecules encoding at least a portion of a protein selected from any one or more, any two or more, any three or more, any four or more, any five or more, any six or more, any seven or more, any eight or more, any nine or more, any ten or more, any eleven or more, any twelve or more, or all thirteen of AKAP2, ARRB1, CPSF7, INO80C, JAG1, MSMP, NR4A2, PLEK, RAP1GAP2, SPEG, TRPS1, UBE2Q1, and ZNF768. This C-RNA signature is identified as list (j) and as described in more detail in WO 2021/102236.

In some embodiments, such a C-RNA signature within the maternal circulation includes C-RNA molecules encoding at least a portion of a protein selected from HCG4P8, EYS, AGGF1P10, and/or GLYATL2 in combination with a plurality of C-RNA molecules encoding at least a portion of a protein selected from of any one or more of any of (a), (b), (c), (d), (e), (f), (g), (h), (i), and/or (j).

The term “plurality” refers to more than one element, for example, the term is used herein in reference to a number of C-RNA molecules that serve as a signature indicative of preeclampsia. A plurality may include any two, any three, any four, any five, any six, any seven, any eight, any nine, any ten, any eleven, any twelve, any thirteen, any fourteen, any fifteen, any sixteen, any seventeen, any eighteen, any nineteen, any twenty, any twenty-one, any twenty-two, any twenty-three, any twenty-four, any twenty-five, any twenty-six, any twenty-seven, any twenty-eight, any twenty-nine, any thirty, any thirty-one, any thirty-two, any thirty-three, any thirty-four, any thirty-five, any thirty-six, any thirty-seven, any thirty-eight, any thirty-nine, any forty, any forty-one, any forty-two, any forty-three, any forty-four, any forty-five, any forty-six, any forty-seven, any forty-eight, any forty-nine, any fifty, any fifty-one, any fifty-two, any fifty-three, any fifty-four, any fifty-five, any fifty-six, any fifty-seven, any fifty-eight, any fifty-nine, any sixty, any sixty-one, any sixty-two, any sixty-three, any sixty-four, any sixty-five, any sixty-six, any sixty-seven, any sixty-eight, any sixty-nine, any seventy, any seventy-one, any seventy-two, any seventy-three, any seventy-four, any seventy-five, any seventy-six, any seventy-seven, any seventy-eight, any seventy-nine, any eighty, any eighty-one, any eighty-two, any eighty-three, any eighty-four, any eighty-five, any eighty-six, any eighty-seven, any eighty-eight, any eighty-nine, any ninety, any ninety-one, any ninety-two, any ninety-three, any ninety-four, any ninety-five, any ninety-six, any ninety-seven, any ninety-eight, any ninety-nine, any one hundred, any one hundred and one, any one hundred and two, any one hundred and three, any one hundred and four, any one hundred and five, any one hundred and six, any one hundred and seven, any one hundred and eight, any one hundred and nine, any one hundred ten, any one hundred eleven, any one hundred twelve, any one hundred thirteen, any one hundred fourteen, any one hundred fifteen, any one hundred sixteen, any one hundred seventeen, any one hundred eighteen, any one hundred nineteen, any one hundred twenty, any one hundred twenty-one, or any one hundred twenty-two of the molecules recited in a list described herein. A plurality may include a least any one of the numbers recited above. A plurality may include more than any one of the numbers recited above. A plurality may include a range of any of those recited above. In some embodiments, a C-RNA signature indicative of preeclampsia includes just one of the biomarkers recited above.

A sample may be a biological sample or biosample, including but not limited to blood, serum, plasma, sweat, tears, urine, sputum, lymph, saliva, amniotic fluid, a tissue biopsy, swab, or smear, including for example, but not limited to, a placental tissue sample. In some preferred embodiments, a biological sample is a cell free plasma sample. A biological sample may be a maternal sample obtained from a pregnant female subject.

With obtaining, shipping, storing, and/or processing blood samples for the preparation of circulating RNA, steps may be taken to stabilize the sample and/or prevent the disruption of cell membranes resulting in the release of cellular RNAs into the sample. For example, in some embodiments, blood samples may be collected, shipped, and/or stored in tubes that have cell- and DNA-stabilizing properties, such as Streck Cell-Free DNA BCT© blood collection tubes, prior to processing into plasma. In some embodiments, blood samples are not exposed to EDTA. See, for example, Qin et al., 2013, BMC Research Notes; 6:380 and Medina Diaz et al., 2016, PLoS ONE; 11(11):e0166354. In some embodiments, blood samples are processed into plasma within about 24 to about 72 hours of the blood draw, and in some embodiments, within about 24 hours of the blood draw. In some embodiments, blood samples are maintained, stored, and/or shipped at room temperature prior to processing into plasma. In some embodiments, blood samples are maintained, stored, and/or shipped without exposure to chilling (for example, on ice) or freezing prior to processing into plasma.

As used herein, the term “subject” refers to a human subject as well as a non-human mammalian subject. Although the examples herein concern humans and the language is primarily directed to human concerns, the concept of this disclosure is applicable to any mammal, and is useful in the fields of veterinary medicine, animal sciences, research laboratories and such.

A subject may be a pregnant female, including a pregnant female in any gestational stages of pregnancy. The gestational stage of pregnancy may be, for example, the first trimester, the second trimester, including late second trimester, or the third trimester, including early third trimester. The gestational stage of pregnancy may be, for example, before about 16 weeks of pregnancy, before about 20 weeks of pregnancy, or after about 20 weeks of pregnancy. The gestational stage of pregnancy may be, for example, about 8 to about 18 weeks of pregnancy, about 10 to about 14 weeks of pregnancy, about 11 to about 14 weeks of pregnancy, about 11 to about 13 weeks, or about 12 to about 13 weeks of pregnancy. The gestational stage of pregnancy may be, for example, about 9 weeks gestation to about 12 weeks gestation, about 13 weeks gestation to about 16 weeks gestation, about 17 weeks gestation to about 20 weeks gestation, about 21 weeks gestation to about 24 weeks gestation, about 25 weeks gestation to about 28 weeks gestation, about 29 weeks gestation to about 32 weeks gestation, about 33 weeks gestation to about 36 weeks gestation, or about 37 weeks gestation to about 40 weeks gestations. The gestational stage of pregnancy may be, for example, about 12 weeks gestation, about 20 weeks gestations, about 28 weeks gestation, or about 36 weeks gestation.

The detection, identification, and/or quantification of C-RNA biomarkers within the maternal circulation associated with a diagnosis of preeclampsia or a risk for developing preeclampsia may involve any of a variety of technologies. For example, biomarkers may be detected in serum by radioimmunoassay or the polymerase chain reaction (PCR) technique may be used.

In various embodiments, the detection, identification, and/or quantification of C-RNA biomarkers in the maternal circulation indicative of preeclampsia or a risk for developing preeclampsia may involve sequencing the C-RNA molecules. Any of a number of sequencing technologies can be utilized, including, but not limited to, any of a variety of high-throughput sequencing techniques.

In some embodiments, the C-RNA population within a maternal biosample may be subject to enrichment of RNA sequences the include protein-coding sequences prior to sequencing. Any of a variety of platforms available for whole-exome enrichment and sequencing may be used, including but not limited to the Agilent SureSelect Human All Exon platform (Chen et al., 2015a, Cold Spring Harb Protoc; 2015(7):626-33. doi: 10.1101/pdb.prot083659); the Roche NimbleGen SeqCap EZ Exome Library SR platform (Chen et al., 2015b, Cold Spring Harb Protoc; 2015(7):634-41. doi: 10.1101/pdb.prot084855); or the Illumina TruSeq Exome Enrichment platform (Chen et al., 2015c, Cold Spring Harb Protoc; 2015(7):642-8. doi:10.1101/pdb.prot084863). See also “TruSeq™ Exome Enrichment Guide,” Catalog #FC-930-1012 Part #15013230 Rev. B November 2010 and Illumina's “TruSeq™ RNA Sample Preparation Guide,” Catalog #RS-122-9001DOC Part #15026495 Rev. F March 2014.

In particular embodiments, C-RNA biomarkers within the maternal circulation indicative of preeclampsia or a risk for developing preeclampsia may be detected, identified, and/or quantified using microarray techniques. In this method, polynucleotide sequences of interest are plated, or arrayed, on a microchip substrate. The arrayed sequences are then hybridized with a maternal biosample, or a purified and/or enriched portion thereof. Microarrays may include a variety of solid supports including, but not limited to, beads, glass microscope slides, glass wafers, gold, silicon, microchips, and other plastic, metal, ceramic, or biological surfaces. Microarray analysis can be performed by commercially available equipment, following manufacturer's protocols, such as by using Illumina's technology.

A biosample obtained from a pregnant female, in addition to containing circulating RNA of maternal origin, will also contain nucleic acids of fetal origin. The discovery of cell-free fetal nucleic acids in maternal plasma has opened up new possibilities for noninvasive prenatal diagnosis. See, for example, Poon et al., 2000, Clin Chem; 1832-4; Poon et al., 2001, Ann N Y Acad Sci; 945:207-10; Ng et al., 2003, Clin Chem; 49(5):727-31; Ng et al., 2003, Proc Natl Acad Sci USA.; 100(8):4748-53; Tsui et al., 2004, J Med Genet; 41(6):461-7; Go et al., 2004, Clin Chem; 50(8):1413-4; Smets et al., 2006, Clin Chim Acta; 364(1-2):22-32; Tsui et al., 2006, Methods Mol Biol; 336:123-34; Purwosunu et al., 2007, Clin Chem; 53(3):399-404; Chim et al., 2008, Clin Chem; 54(3):482-90; Tsui and Lo, 2008, Methods Mol Biol; 444:275-89; Lo, 2008, Ann N Y Acad Sci; 1137:140-143; Miura et al., 2010, Prenat Diagn; 30(9):849-61; Li et al., 2012, Clin Chim Acta; 413(5-6):568-76; Williams et al., 2013, Proc Natl Acad Sci USA; 110(11):4255-60; Tsui et al., 2014, Clin Chem; 60(7):954-62; Tsang et al., 2017, Proc Natl Acad Sci USA; 114(37):E7786-E7795, and US Patent Publication US 2014/0243212.

Prenatal testing in recent years has been moving towards non-invasive methods to determine the fetal risk for genetic disorders. Cell-free fetal DNA (cffDNA) is extracellular DNA of fetal origin that is found in the maternal circulation in a fraction ranging about 2-10% of the total DNA in maternal blood. Noninvasive prenatal testing (NIPT) for fetal chromosome abnormalities using cell-free DNA (cf DNA) in the maternal circulation became clinically available in the United States in October 2011. Assaying the cell-free fetal DNA present in maternal plasma via various molecular methods is now used to identify a range of fetal chromosomal aneuploidies, such as, for example, trisomy 21, trisomy 13, and trisomy 18, determine the sex of the fetus, and identify various gene mutations, such as, for example, Tay-Sachs disease, sickle cell anemia, thalassemia, cystic fibrosis, muscular dystrophy, and fragile X syndrome. Noninvasive prenatal testing for fetal chromosome abnormalities using cell-free DNA in the maternal circulation may be for screening purposes or for diagnostic purposes.

In some embodiments, the detection, identification, and/or quantification of C-RNA biomarkers within the maternal circulation indicative of preeclampsia or a risk for developing preeclampsia as described herein may be combined with prenatal genetic testing, including also assaying a biosample for cell free fetal DNA within maternal circulation to determine the health and condition of an unborn fetus. For example, a biosample may be assayed for both C-RNA biomarkers indicative of preeclampsia or a risk for developing preeclampsia and for cell-free fetal DNA for fetal chromosome abnormalities. Or, one biosample may be divided into portions, with one portion assayed for C-RNA biomarkers indicative of preeclampsia or a risk for developing preeclampsia and another portion assayed for cell-free fetal DNA for fetal chromosome abnormalities. Any of a variety of available methods for assaying cell-free fetal DNA for fetal chromosome abnormalities may be utilized.

The disclosure includes kits for use in the diagnosis of preeclampsia and the identification of pregnant women at risk for developing preeclampsia. A kit is any manufacture (for example, a package or container) including at least one reagent (for example, a probe), for specifically detecting a C-RNA signature within the maternal circulation as described herein that is indicative of preeclampsia or a risk for developing preeclampsia. The kit may be promoted, distributed, or sold as a unit for performing the methods of the present disclosure.

The use of circulating RNA biomarkers found in the maternal circulation specific to preeclampsia in noninvasive methods for the diagnosis of preeclampsia and the identification of pregnant women at risk for developing preeclampsia may be combined with appropriate monitoring and medical management. For example, further tests may be ordered. Such test may include, for example, blood tests to measure liver function, kidney function, and/or platelet and various clotting proteins, urine analysis to measure protein or creatinine levels, fetal ultrasound to measure monitor fetal growth, weight, and amniotic fluid, a nonstress test to measure how fetal heart rate with fetal movement, and/or a biophysical profile using ultrasound to measure your fetal breathing, muscle tone, and movement and the volume of amniotic fluid may be ordered. Therapeutic interventions may include, for example, increasing the frequency of prenatal visits, antihypertensive medications to lower blood pressure, corticosteroid medications, anticonvulsant medications, bed rest, hospitalization, and/or preterm delivery. See, for example, Townsend et al., 2016 “Current best practice in the management of hypertensive disorders in pregnancy,” Integr Blood Press Control; 9:79-94.

Therapeutic interventions may include the administration of low dose aspirin to pregnant women identified at risk of for developing preeclampsia. A recent multicenter, double-blind, placebo-controlled trial demonstrated that treatment of women at high risk for preterm preeclampsia with low-dose aspirin resulted in a lower incidence of this diagnosis compared to placebo (Rolnik et al., 2017, “Aspirin versus Placebo in Pregnancies at High Risk for Preterm Preeclampsia,” N Engl J Med; 377(7):613-622). Dosages of low dose aspirin include, but are not limited to, about 50 to about 150 mg per day, about 60 to about 80 mg per day, about 100 or more mg per day, or about 150 mg per day. Administration may begin, for example, at or before 16 weeks of gestation or from 11 to 14 weeks of gestation. Administration may continue thru 36 weeks of gestation.

### EMBODIMENTS

The invention is defined in the claims. However, below there is provided a non-exhaustive listing of non-limiting exemplary aspects. Any one or more of the features of these aspects may be combined with any one or more features of another example, embodiment, or aspect described herein. Exemplary Embodiments of the present invention include, but are not limited to, the following.

Aspect 1 includes a method of detecting preeclampsia and/or determining an increased risk for preeclampsia in a pregnant female, the method comprising:
identifying in a biosample obtained from the pregnant female a circulating RNA (C-RNA) molecule encoding at least a portion of a protein selected from any one or more, any two or more, any three or more, or all four of HCG4P8, EYS, AGGF1P10, and GLYATL2;
wherein identifying the C-RNA molecule encoding at least a portion of a protein selected from any one or more, any two or more, any three or more, or all four of HCG4P8, EYS, AGGF1P10, and GLYATL2 is indicative of preeclampsia and/or an increased risk for preeclampsia in the pregnant female.

Aspect 2 includes a method of detecting preeclampsia and/or determining an increased risk for preeclampsia in a pregnant female, the method comprising:
purifying a population of circulating RNA (C-RNA) molecules from a biosample obtained from the pregnant female;
identifying protein coding sequences encoded by the C-RNA molecules within the purified population of C-RNA molecules;
wherein the identification of protein coding sequences encoded by the C-RNA molecules encoding at least a portion of a protein are selected from any one or more, any two or more, any three or more, or all four of HCG4P8, EYS, AGGF1P10, and GLYATL2 is indicative of preeclampsia and/or an increased risk for preeclampsia in the pregnant women.

Aspect 3 includes the method of Aspects 1 or 2, wherein identifying protein coding sequences encoded by the C-RNA molecules within the biosample comprises hybridization, reverse transcriptase PCR, microarray chip analysis, or sequencing.

Aspect 4 includes the method of Aspect 3, wherein sequencing comprises clonal amplification and massively parallel sequencing of clonally amplified molecules.

Aspect 5 includes the method of Aspects 3 or 4, wherein sequencing comprises RNA sequencing.

Aspect 6 includes the method of any one of Aspects 2 to 5, further comprising:
removing intact cells from the biosample;
treating the biosample with a deoxynuclease (DNase) to remove cell free DNA (cfDNA;
synthesizing complementary DNA (cDNA) from C-RNA molecules in the biosample; and/or
enriching the cDNA sequences for sequences that encode proteins;
prior to identifying protein coding sequence encoded by the circulating RNA (C-RNA) molecules.

Aspect 7 includes a method of detecting preeclampsia and/or determining an increased risk for preeclampsia in a pregnant female, the method comprising:
removing intact cells from a biosample obtained from the pregnant female;
treating the biosample with a deoxynuclease (DNase) to remove cell free DNA (cfDNA);
synthesizing complementary DNA (cDNA) from RNA molecules in the biosample;
enriching the cDNA sequences for DNA sequences that encode proteins;
sequencing the resulting enriched cDNA sequences; and
identifying protein coding sequences encoded by enriched cDNA sequences;
wherein the identification of protein coding sequences encoded by the enriched cDNA sequences encoding at least a portion of a protein selected from any one or more, any two or more, any three or more, or all four of HCG4P8, EYS, AGGF1P10, and GLYATL2 is indicative of preeclampsia and/or an increased risk for preeclampsia in the pregnant women.

Aspect 8 includes the method of any one of Aspects 1 to 7, wherein detecting preeclampsia and/or determining an increased risk for preeclampsia comprises detecting early preeclampsia and/or determining an increased risk for early preeclampsia.

Aspect 9 includes a method comprising:
removing intact cells from a biosample obtained from a pregnant female;
treating the biosample with a deoxynuclease (DNase) to remove cell free DNA (cfDNA);
synthesizing complementary DNA (cDNA) sequences from RNA molecules in the biosample;
enriching the cDNA sequences for DNA sequences that encode proteins;
sequencing the resulting enriched cDNA sequences; and
identifying within the resulting protein coding sequences encoded by the enriched C-RNA molecules protein coding sequences including at least a portion of a protein selected from any one or more, any two or more, any three or more, or all four of HCG4P8, EYS, AGGF1P10, and GLYATL2.

Aspect 10 includes the method of any one of Aspects 1 to 9, wherein the biosample is obtained from a pregnant female at about 9 weeks gestation to about 12 weeks gestation, about 13 weeks gestation to about 16 weeks gestation, about 17 weeks gestation to about 20 weeks gestation, about 21 weeks gestation to about 24 weeks gestation, about 25 weeks gestation to about 28 weeks gestation, about 29 weeks gestation to about 32 weeks gestation, about 33 weeks gestation to about 36 weeks gestation, or about 37 weeks gestation to about 40 weeks gestations.

Aspect 11 includes the method of any one of Aspects 1 to 10, wherein the biosample is obtained from a pregnant female at about 12 weeks gestation, about 20 weeks gestations, about 28 weeks gestation, or about 36 weeks gestation.

Aspect 12 includes the method of any one of Aspects 1 to 11, wherein the biosample comprises plasma.

Aspect 13 includes the method of any one of Aspects 1 to 11, wherein sample is a blood sample and the blood sample is:
not exposed to EDTA prior to processing the blood sample into plasma;
processed into plasma within about 24 to about 72 hours of the blood draw;
maintained, stored, and/or shipped at room temperature prior to processing into plasma; and/or
maintained, stored, and/or shipped without exposure to chilling or freezing prior to processing into plasma.

Aspect 14 includes the method of any one of Aspects 1 to 13, wherein:
HCG4P8 is downregulated in comparison to a normal control;
EYS is downregulated in comparison to a normal control;
AGGF1P10 is downregulated in comparison to a normal control; and/or
GLYATL2 is upregulated in comparison to a normal control.

Aspect 15 includes the method of any one of Aspects 1 to 14, further comprising identifying within the biosample a circulating RNA (C-RNA) molecule encoding at least a portion of a protein selected from:
(a) any one or more, any two or more, any three or more, any four or more, any five or more, any six or more, any seven or more, any eight or more, any nine or more, any ten or more, any eleven or more, any twelve, any thirteen or more, any fourteen or more, any fifteen or more, any sixteen or more, any seventeen or more, any eighteen or more, any nineteen or more, any twenty or more, any twenty one or more, any twenty two or more, any twenty three or more, any twenty four or more, any twenty five or more, any fifty or more, any seventy or more, or all seventy-five of ARRDC2, JUN, SKIL, ATP13A3, PDE8B, GSTA3, PAPPA2, TIPARP, LEP, RGP1, USP54, CLEC4C, MRPS35, ARHGEF25, CUX2, HEATR9, FSTL3, DDI2, ZMYM6, ST6GALNAC3, GBP2, NES, ETV3, ADAM17, ATOH8, SLC4A3, TRAF3IP1, TTC21A, HEG1, ASTE1, TMEM108, ENC1, SCAMPI, ARRDC3, SLC26A2, SLIT3, CLIC5, TNFRSF21, PPP1R17, TPST1, GATSL2, SPDYE5, HIPK2, MTRNR2L6, CLCN1, GINS4, CRH, C10orf2, TRUB1, PRG2, ACY3, FAR2, CD63, CKAP4, TPCN1, RNF6, THTPA, FOS, PARN, ORAI3, ELMO3, SMPD3, SERPINF1, TMEM11, PSMD11, EBI3, CLEC4M, CCDC151, CPAMD8, CNFN, LILRA4, ADA, C22orf39, PI4KAP1, and ARFGAP3; or
(b) any one or more, any two or more, any three or more, any four or more, any five or more, any six or more, any seven or more, any eight or more, any nine or more, any ten or more, any eleven or more, any twelve or more, any thirteen or more, any fourteen or more, any fifteen or more, any sixteen or more, any seventeen or more, any eighteen or more, any nineteen or more, any twenty or more, any twenty one or more, any twenty two or more, any twenty three or more, any twenty four or more, any twenty five or more, any twenty six of more, or all twenty-seven of TIMP4, FLG, HTRA4, AMPH, LCN6, CRH, TEAD4, ARMS2, PAPPA2, SEMA3G, ADAMTS1, ALOX15B, SLC9A3R2, TIMP3, IGFBP5, HSPA12B, CLEC4C, KRT5, PRG2, PRX, ARHGEF25, ADAMTS2, DAAM2, FAM107A, LEP, NES, and VSIG4; or
(c) any one or more, any two or more, any three or more, any four or more, any five or more, any six or more, any seven or more, any eight or more, any nine or more, any ten or more, any eleven or more, any twelve, any thirteen or more, any fourteen or more, any fifteen or more, any sixteen or more, any seventeen or more, any eighteen or more, any nineteen or more, any twenty or more, any twenty one or more, any twenty two or more, any twenty three or more, any twenty four or more, any twenty five or more, any fifty or more, any seventy-five or more, any one hundred or more, or all one hundred twenty-two of CYP26B1, IRF6, MYH14, PODXL, PPP1R3C, SH3RF2, TMC7, ZNF366, ADCY1, C6, FAM219A, HAO2, IGIP, IL1R2, NTRK2, SH3PXD2A, SSUH2, SULT2A1, FMO3, FSTL3, GATA5, HTRA1, C8B, H19, MN1, NFE2L1, PRDM16, AP3B2, EMP1, FLNC, STAG3, CPB2, TENC1, RP1L1, A1CF, NPR1, TEK, ERRFIl, ARHGEF15, CD34, RSPO3, ALPK3, SAMD4A, ZCCHC24, LEAP2, MYL2, NRG3, ZBTB16, SERPINA3, AQP7, SRPX, UACA, ANO1, FKBP5, SCN5A, PTPN21, CACNAlC, ERG, SOX17, WWTR1, AIF1L, CA3, HRG, TAT, AQP7P1, ADRA2C, SYNPO, FN1, GPR116, KRT17, AZGP1, BCL6B, KIFIC, CLIC5, GPR4, GJA5, OLAH, C14orf37, ZEB1, JAG2, KIF26A, APOLD1, PNMT, MYOM3, PITPNM3, TIMP4, HTRA4, AMPH, LCN6, CRH, TEAD4, ARMS2, PAPPA2, SEMA3G, ADAMTS1, ALOX15B, SLC9A3R2, TIMP3, IGFBP5, HSPA12B, PRG2, PRX, ARHGEF25, ADAMTS2, DAAM2, FAM107A, LEP, NES, VSIG4, HBG2, CADM2, LAMP5, PTGDR2, NOMO1, NXF3, PLD4, BPIFB3, PACSIN1, CUX2, FLG, CLEC4C, and KRT5; or
(d) any one or more, any two or more, any three or more, any four or more, any five or more, any six or more, any seven or more, any eight or more, any nine or more, any ten or more, any eleven or more, any twelve or more, any thirteen or more, any fourteen or more, any fifteen or more, any sixteen or more, any seventeen or more, any eighteen or more, any nineteen or more, any twenty or more, any twenty-one or more, any twenty-two or more, any twenty-three or more, any twenty-four or more, any twenty-five or more, any twenty-six or more, any twenty-seven or more, any twenty-eight or more, any twenty-nine or more, or all thirty of VSIG4, ADAMTS2, NES, FAM107A, LEP, DAAM2, ARHGEF25, TIMP3, PRX, ALOX15B, HSPA12B, IGFBP5, CLEC4C, SLC9A3R2, ADAMTS1, SEMA3G, KRT5, AMPH, PRG2, PAPPA2, TEAD4, CRH, PITPNM3, TIMP4, PNMT, ZEB1, APOLD1, PLD4, CUX2, and HTRA4; or
(e) any one or more, any two or more, any three or more, any four or more, any five or more, any six or more, any seven or more, any eight or more, any nine or more, any ten or more, any eleven or more, any twelve or more, any thirteen or more, any fourteen or more, any fifteen or more, any sixteen or more, any seventeen or more, any eighteen or more, any nineteen or more, any twenty or more, any twenty-one or more, any twenty-two or more, any twenty-three or more, any twenty-four or more, any twenty-five or more, or all twenty-six of ADAMTS1, ADAMTS2, ALOX15B, AMPH, ARHGEF25, CELF4, DAAM2, FAM107A, HSPA12B, HTRA4, IGFBP5, KCNA5, KRT5, LCN6, LEP, LRRC26, NES, OLAH, PACSIN1, PAPPA2, PRX, PTGDR2, SEMA3G, SLC9A3R2, TIMP3, and VSIG4; or
(f) any one or more, any two or more, any three or more, any four or more, any five or more, any six or more, any seven or more, any eight or more, any nine or more, any ten or more, any eleven or more, any twelve or more, any thirteen or more, any fourteen or more, any fifteen or more, any sixteen or more, any seventeen or more, any eighteen or more, any nineteen or more, any twenty or more, any twenty-one or more, or all twenty-two of ADAMTS1, ADAMTS2, ALOX15B, ARHGEF25, CELF4, DAAM2, FAM107A, HTRA4, IGFBP5, KCNA5, KRT5, LCN6, LEP, LRRC26, NES, OLAH, PRX, PTGDR2, SEMA3G, SLC9A3R2, TIMP3, and VSIG4; or
(g) any one or more, any two or more, any three or more, any four or more, any five or more, any six or more, any seven or more, any eight or more, any nine or more, any ten or more, or all eleven of CLEC4C, ARHGEF25, ADAMTS2, LEP, ARRDC2, SKIL, PAPPA2, VSIG4, ARRDC4, CRH, and NES; or
(h) any one or more, any two or more, any three or more, any four or more, any five or more, any six or more, any seven or more, any eight or more, any nine or more, any ten or more, any eleven or more, any twelve or more, any thirteen or more, any fourteen or more, any fifteen or more, any sixteen or more, any seventeen or more, any eighteen or more, any nineteen or more, any twenty or more, any twenty-one or more, any twenty-two or more, any twenty-three or more, or all twenty-four of LEP, PAPPA2, KCNA5, ADAMTS2, MYOM3, ATP13A3, ARHGEF25, ADA, HTRA4, NES, CRH, ACY3, PLD4, SCT, NOX4, PACSIN1, SERPINF1, SKIL, SEMA3G, TIPARP, LRRC26, PHEX, LILRA4, and PER1; or
(i) any one or more, any two or more, any three or more, any four or more, any five or more, any six or more, any seven or more, any eight or more, any nine or more, any ten or more, any eleven or more, any twelve, any thirteen or more, any fourteen or more, any fifteen or more, any sixteen or more, any seventeen or more, any eighteen or more, any nineteen or more, any twenty or more, any twenty one or more, any twenty two or more, any twenty three or more, any twenty four or more, any twenty five or more, any twenty-six or more, any twenty-seven or more, any twenty-eight or more, any twenty-nine or more, any thirty or more, any thirty-one or more, any thirty-two or more, any thirty-three or more, any thirty-four or more, any thirty-five or more, any thirty-six or more, any thirty-seven or more, any thirty-eight or more, any thirty-nine or more, any forty or more, any forty-one or more, any forty-two or more, any forty-three or more, any forty-four or more, any forty-five or more, any forty-six or more, any forty-seven or more, any forty-eight or more, or all forth-nine of ADA, ADAMTS2, AKAP2, ARHGEF25, ARRB1, ARRDC2, ATOH8, CLEC4C, CPSF7, CUX2, FKBP5, FSTL3, GSTA3, HEG1, IGIP, INO80C, JAG1, JUN, KRT5, LEP, LILRA4, MRPS35, MSMP, NES, NFE2L1, NR4A2, NTRK2, PACSIN1, PER1, PLD4, PLEK, PRG2, RAP1GAP2, RGP1, SEMA3G, SH3PXD2A, SKIL, SMPD3, SPEG, SRPX, SYNPO, TEAD4, TIPARP, TNFRSF21, TPST1, TRPS1, UBE2Q1, VSIG4, and ZNF768; or
(j) any one or more, any two or more, any three or more, any four or more, any five or more, any six or more, any seven or more, any eight or more, any nine or more, any ten or more, any eleven or more, any twelve or more, or all thirteen of AKAP2, ARRB1, CPSF7, INO80C, JAG1, MSMP, NR4A2, PLEK, RAP1GAP2, SPEG, TRPS1, UBE2Q1, and ZNF768.

Aspect 16 includes the method of any one of Aspects 1 to 15, further comprising performing prenatal genetic screening testing or prenatal genetic diagnostic testing on a portion of the biosample obtained from a pregnant female.

Aspect 17 includes a circulating RNA (C-RNA) signature for preeclampsia or for an elevated risk of preeclampsia, the C-RNA signature comprising any one or more, any two or more, any three or more, or all four of HCG4P8, EYS, AGGF1P10, and GLYATL2.

Aspect 18 includes a solid support array comprising a plurality of agents capable of binding and/or identifying a C-RNA signature of Aspect 17.

Aspect 19 includes a kit comprising a plurality of probes capable of binding and/or identifying a C-RNA signature of Aspect 17.

Aspect 20 includes a kit comprising a plurality of primers for selectively amplifying a C-RNA signature of Aspect 17.

The present invention is illustrated by the following examples. It is to be understood that the particular examples, materials, amounts, and procedures are to be interpreted broadly in accordance with the scope and spirit of the invention as set forth herein.

### EXAMPLES

### Example 1

### Pregnancy Outcome Prediction Study

With several minor modifications, using methods described in more detail in WO 2019/22701, WO 2021/102236, and Munchel et al., 2020, Sci Transl Med; 12(550):eaaz0131. doi: 10.1126/scitranslmed.aaz0131, a preliminary differential expression analysis of Phase 1 of the Pregnancy Outcome Prediction (POP), analyzing early-onset PE samples and 60 matched controls found four transcripts to be associated with preeclampsia (HCG4P8, EYS, AGGF1P10, and GLYATL2). The modifications included modifications to sample preparation when preparing samples and analyzing data for this cohort. Rather than TruSeq, the Illumina RNA Preparation with Enrichment kit was used to prepare sequencing libraries and enrich for transcriptomic sequences from C-RNA. Both approaches have been shown to generate comparable data (see Illumina's “Improved Detection of Circulating Transcripts,” available on the worldwide web at illumina.com/content/dam/illumina/gcs/assembled-assets/marketing-literature/illumina-rna-enrichment-crna-app-note-470-2020-009/illumina-rna-enrichment-crna-app-note-470-2020-009.pdf). Samples were sequenced to a minimum of 100M mapped reads, alignment and transcript counts were generated with the DRAGEN RNA application. Differential expression analysis was performed in edgeR, using a Generalized Linear Model with blocking by sample gestational age to specifically compare preeclampsia and control transcriptome profiles.

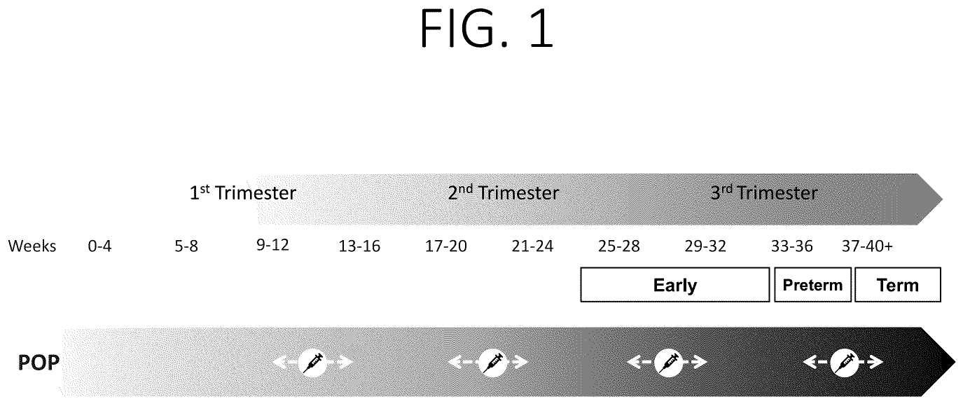

The Pregnancy Outcome Prediction (POP) Study followed 4,500 nulliparous pregnancies, with four blood draws per pregnancy. An overview of the POP Study is show in FIG. 1. Phase 1 of the POP Study has been fully processed, with 752 samples from 195 pregnancies. The pregnancies of Phase 1 included 15 pregnancies with early-onset preeclampsia (PE) with fetal growth restriction (FGR) and preterm delivery and 20 pregnancies with PE with FGR and full term delivery.

### Sample Sizes:

The 12 week timepoint included samples from 15 PE cases and 60 matched controls
The 20 week timepoint included samples from 14 PE cases and 60 matched controls
The 28 week timepoint included samples from 12 PE cases and 60 matched controls
The 36 week timepoint included a sample from 1 case and 54 matched controls. Given the limited number of PE cases at 36 weeks, a DEX analysis was not run for the 36 week timepoint.

Differential expression analysis of cell free RNA comparing longitudinal time course data from pregnant subjects with early-onset preeclampsia with FGR and preterm delivery to control pregnancies identified four altered transcripts. Three are downregulated in the disease and one was upregulated. Two of the transcripts may be related to immune dysfunction, one may relate to fetal development, and one may relate to placental development, all functions impaired in this condition. All transcripts appear to show a difference even at the earliest timepoint (though to of a varying strength), prior to disease symptom onset. One of these transcripts also appears to be altered in the same pattern across two independent cohorts of patients, which supports that the alteration is likely to be a valid marker of this disease (only two transcripts were assessed in this comparison).

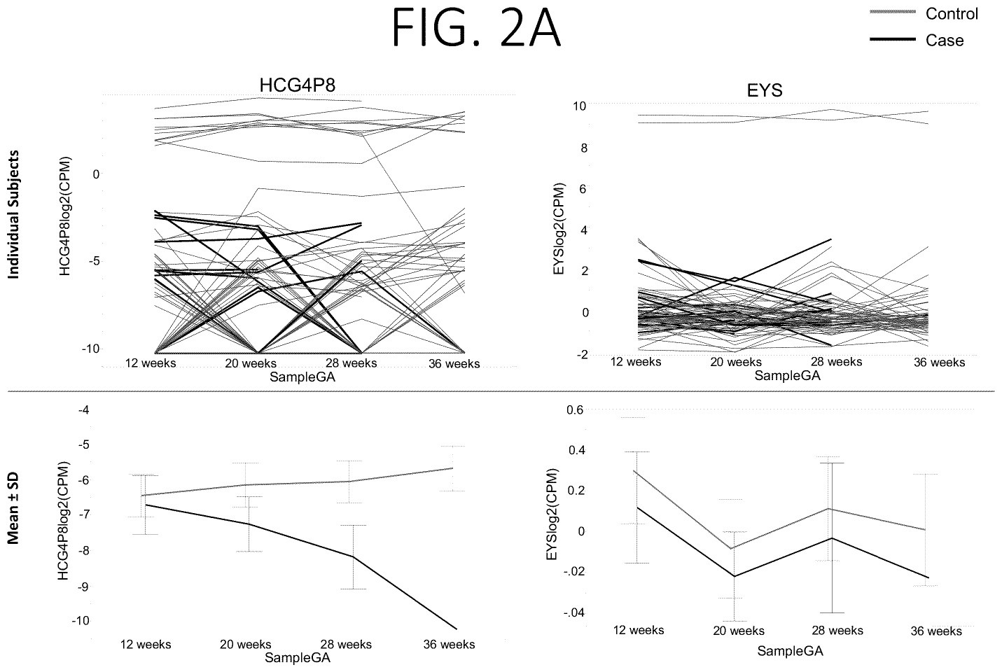

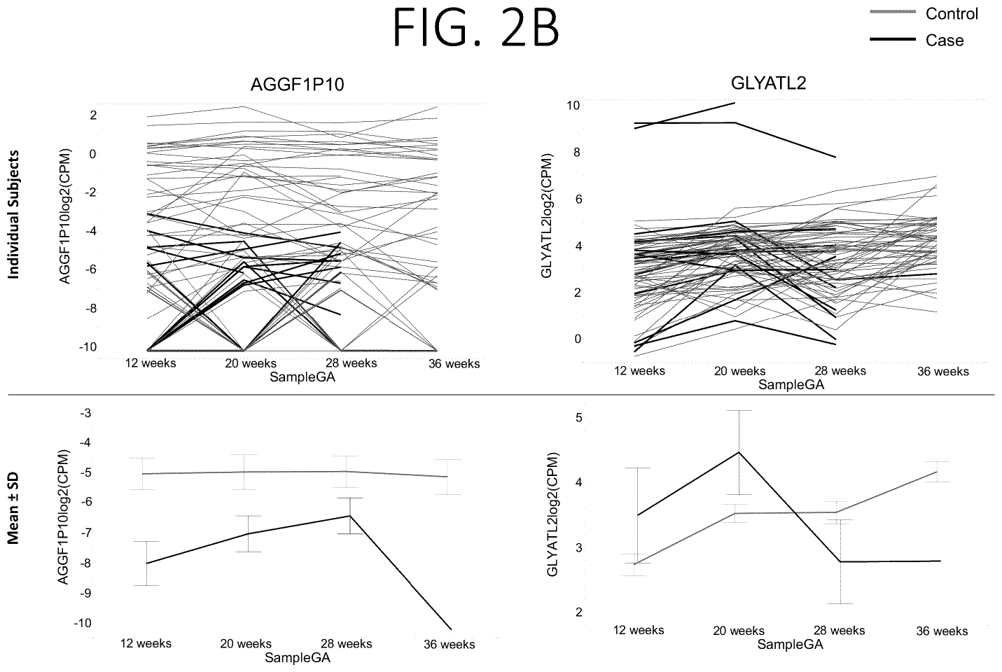

FIGS. 2A and 2B presents an analysis of the DEX transcript data from Phase 1 early-onset PE samples and 60 matched controls. As shown in FIGS. 2A and 2B, HCG4P8, EYS, and AGGF1P10 are down regulated in PE samples and GLYATL2 is upregulated.

HCG4P8, also known as HLA Complex Group 4 Pseudogene 8 (HGNC: 22927; NCBI Entrez Gene: 353005; Ensembl: ENSG00000229142) is an HLA complex pseudogene. EYS, also known as Eyes Shut Homolog (HGNC: 215555; ENSG00000188107) is an epidermal growth factor-like protein that maintains photoreceptor cells and plays a role in protein trafficking. AGGF1P10, also known as Angiogenic Factor With G-Patch And FHA Domains 1 Pseudogene 10 (HGNC: 51747; NCBI Entrez Gene: 100288774; Ensembl: ENSG00000282968) is an angiogenesis factor pseudogene. GLYATL2, also known as Glycine-N-Acyltransferase Like 2 (HGNC: 24178; NCBI Entrez Gene 219970; Ensembl: ENSG00000156689; OMIM®: 614762; UniProtKB/Swiss-Prot: Q8WU03) enables glycine N-acyltransferase activity and is involved in lipid catabolism and signaling.

Reviewing the data of FIGS. 2A and 2B, the PE samples are clearly and consistently at the low end of the distribution, especially at the earliest timepoint. AGGF1P10 trend is quite compelling, with AGGF1P10 convincingly lower in PE samples. GLYATL2 is also demonstrates an interesting trend, being higher at early timepoints and lower at later ones. HCG4P8 and EYS may have influence from outlier control samples and additional follow is warranted.

The present results were compared to results from an unrelated orthogonal cohort which included 6 samples with preeclampsia and preterm birth, 7 samples with any preterm birth, and 72 samples with no preeclampsia or preterm birth. Comparing the present results with the results of this unrelated study, there is a potential GLYATL2 corroboration. GLYATL2 shows the same pattern of change in both studies. GLYATL2 starts out elevated in PE subjects through 20 weeks, then drops below control level by 28 weeks in both studies. Healthy control pregnancies increase consistently for both studies.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. Supplementary materials referenced in publications (such as supplementary tables, supplementary figures, supplementary materials, and methods, and/or supplementary experimental data) are likewise incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The disclosure is not limited to the exact details shown and described, for variations obvious to one skilled in the art will be included within the disclosure defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present disclosure. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the disclosure are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

What is claimed is:

1. A method of detecting preeclampsia and/or determining an increased risk for preeclampsia in a pregnant female, the method comprising:
identifying in a biosample obtained from the pregnant female a circulating RNA (C-RNA) molecule encoding at least a portion of a protein selected from any one or more, any two or more, any three or more, or all four of HCG4P8, EYS, AGGF1P10, and GLYATL2;
wherein identifying the C-RNA molecule encoding at least a portion of a protein selected from any one or more, any two or more, any three or more, or all four of HCG4P8, EYS, AGGF1P10, and GLYATL2 is indicative of preeclampsia and/or an increased risk for preeclampsia in the pregnant female.

2. A method of detecting preeclampsia and/or determining an increased risk for preeclampsia in a pregnant female, the method comprising:
purifying a population of circulating RNA (C-RNA) molecules from a biosample obtained from the pregnant female;
identifying protein coding sequences encoded by the C-RNA molecules within the purified population of C-RNA molecules;
wherein the identification of protein coding sequences encoded by the C-RNA molecules encoding at least a portion of a protein are selected from any one or more, any two or more, any three or more, or all four of HCG4P8, EYS, AGGF1P10, and GLYATL2 is indicative of preeclampsia and/or an increased risk for preeclampsia in the pregnant women.

3. The method of claim 1, wherein identifying protein coding sequences encoded by the C-RNA molecules within the biosample comprises hybridization, reverse transcriptase PCR, microarray chip analysis, or sequencing.

4. The method of claim 3, wherein sequencing comprises clonal amplification and massively parallel sequencing of clonally amplified molecules.

5. The method of claim 3, wherein sequencing comprises RNA sequencing.

6. The method of claim 2, further comprising:
removing intact cells from the biosample;
treating the biosample with a deoxynuclease (DNase) to remove cell free DNA (cfDNA;
synthesizing complementary DNA (cDNA) from C-RNA molecules in the biosample; and/or
enriching the cDNA sequences for sequences that encode proteins;
prior to identifying protein coding sequence encoded by the circulating RNA (C-RNA) molecules.

7. The method of claim 1, wherein detecting preeclampsia and/or determining an increased risk for preeclampsia comprises detecting early preeclampsia and/or determining an increased risk for early preeclampsia.

8. A method comprising:
removing intact cells from a biosample obtained from a pregnant female;
treating the biosample with a deoxynuclease (DNase) to remove cell free DNA (cfDNA);
synthesizing complementary DNA (cDNA) sequences from RNA molecules in the biosample;
enriching the cDNA sequences for DNA sequences that encode proteins;
sequencing the resulting enriched cDNA sequences; and
identifying within the resulting protein coding sequences encoded by the enriched C-RNA molecules protein coding sequences including at least a portion of a protein selected from any one or more, any two or more, any three or more, or all four of HCG4P8, EYS, AGGF1P10, and GLYATL2.

9. The method of claim 1, wherein the biosample is obtained from a pregnant female at about 9 weeks gestation to about 12 weeks gestation, about 13 weeks gestation to about 16 weeks gestation, about 17 weeks gestation to about 20 weeks gestation, about 21 weeks gestation to about 24 weeks gestation, about 25 weeks gestation to about 28 weeks gestation, about 29 weeks gestation to about 32 weeks gestation, about 33 weeks gestation to about 36 weeks gestation, or about 37 weeks gestation to about 40 weeks gestations.

10. The method of claim 1, wherein the biosample is obtained from a pregnant female at about 12 weeks gestation, about 20 weeks gestations, about 28 weeks gestation, or about 36 weeks gestation.

11. The method of claim 1, wherein the biosample comprises plasma.

12. The method of claim 1, wherein sample is a blood sample and the blood sample is:
not exposed to EDTA prior to processing the blood sample into plasma;
processed into plasma within about 24 to about 72 hours of the blood draw;
maintained, stored, and/or shipped at room temperature prior to processing into plasma; and/or
maintained, stored, and/or shipped without exposure to chilling or freezing prior to processing into plasma.

13. The method of claim 1, wherein:
HCG4P8 is downregulated in comparison to a normal control;
EYS is downregulated in comparison to a normal control;
AGGF1P10 is downregulated in comparison to a normal control; and/or
GLYATL2 is upregulated in comparison to a normal control.

14. The method of claim 1, further comprising identifying within the biosample a circulating RNA (C-RNA) molecule encoding at least a portion of a protein selected from:
(a) any one or more, any two or more, any three or more, any four or more, any five or more, any six or more, any seven or more, any eight or more, any nine or more, any ten or more, any eleven or more, any twelve, any thirteen or more, any fourteen or more, any fifteen or more, any sixteen or more, any seventeen or more, any eighteen or more, any nineteen or more, any twenty or more, any twenty one or more, any twenty two or more, any twenty three or more, any twenty four or more, any twenty five or more, any fifty or more, any seventy or more, or all seventy-five of ARRDC2, JUN, SKIL, ATP13A3, PDE8B, GSTA3, PAPPA2, TIPARP, LEP, RGP1, USP54, CLEC4C, MRPS35, ARHGEF25, CUX2, HEATR9, FSTL3, DDI2, ZMYM6, ST6GALNAC3, GBP2, NES, ETV3, ADAM17, ATOH8, SLC4A3, TRAF3IP1, TTC21A, HEG1, ASTE1, TMEM108, ENC1, SCAMPI, ARRDC3, SLC26A2, SLIT3, CLIC5, TNFRSF21, PPP1R17, TPST1, GATSL2, SPDYE5, HIPK2, MTRNR2L6, CLCN1, GINS4, CRH, C10orf2, TRUB1, PRG2, ACY3, FAR2, CD63, CKAP4, TPCN1, RNF6, THTPA, FOS, PARN, ORAI3, ELMO3, SMPD3, SERPINF1, TMEM11, PSMD11, EBI3, CLEC4M, CCDC151, CPAMD8, CNFN, LILRA4, ADA, C22orf39, PI4KAP1, and ARFGAP3; or
(b) any one or more, any two or more, any three or more, any four or more, any five or more, any six or more, any seven or more, any eight or more, any nine or more, any ten or more, any eleven or more, any twelve or more, any thirteen or more, any fourteen or more, any fifteen or more, any sixteen or more, any seventeen or more, any eighteen or more, any nineteen or more, any twenty or more, any twenty one or more, any twenty two or more, any twenty three or more, any twenty four or more, any twenty five or more, any twenty six of more, or all twenty-seven of TIMP4, FLG, HTRA4, AMPH, LCN6, CRH, TEAD4, ARMS2, PAPPA2, SEMA3G, ADAMTS1, ALOX15B, SLC9A3R2, TIMP3, IGFBP5, HSPA12B, CLEC4C, KRT5, PRG2, PRX, ARHGEF25, ADAMTS2, DAAM2, FAM107A, LEP, NES, and VSIG4; or
(c) any one or more, any two or more, any three or more, any four or more, any five or more, any six or more, any seven or more, any eight or more, any nine or more, any ten or more, any eleven or more, any twelve, any thirteen or more, any fourteen or more, any fifteen or more, any sixteen or more, any seventeen or more, any eighteen or more, any nineteen or more, any twenty or more, any twenty one or more, any twenty two or more, any twenty three or more, any twenty four or more, any twenty five or more, any fifty or more, any seventy-five or more, any one hundred or more, or all one hundred twenty-two of CYP26B1, IRF6, MYH14, PODXL, PPP1R3C, SH3RF2, TMC7, ZNF366, ADCY1, C6, FAM219A, HAO2, IGIP, IL1R2, NTRK2, SH3PXD2A, SSUH2, SULT2A1, FMO3, FSTL3, GATA5, HTRA1, C8B, H19, MN1, NFE2L1, PRDM16, AP3B2, EMP1, FLNC, STAG3, CPB2, TENC1, RP1L1, A1CF, NPR1, TEK, ERRFIl, ARHGEF15, CD34, RSPO3, ALPK3, SAMD4A, ZCCHC24, LEAP2, MYL2, NRG3, ZBTB16, SERPINA3, AQP7, SRPX, UACA, ANO1, FKBP5, SCN5A, PTPN21, CACNAlC, ERG, SOX17, WWTR1, AIF1L, CA3, HRG, TAT, AQP7P1, ADRA2C, SYNPO, FN1, GPR116, KRT17, AZGP1, BCL6B, KIFIC, CLIC5, GPR4, GJA5, OLAH, C14orf37, ZEB1, JAG2, KIF26A, APOLD1, PNMT, MYOM3, PITPNM3, TIMP4, HTRA4, AMPH, LCN6, CRH, TEAD4, ARMS2, PAPPA2, SEMA3G, ADAMTS1, ALOX15B, SLC9A3R2, TIMP3, IGFBP5, HSPA12B, PRG2, PRX, ARHGEF25, ADAMTS2, DAAM2, FAM107A, LEP, NES, VSIG4, HBG2, CADM2, LAMP5, PTGDR2, NOMO1, NXF3, PLD4, BPIFB3, PACSIN1, CUX2, FLG, CLEC4C, and KRT5; or
(d) any one or more, any two or more, any three or more, any four or more, any five or more, any six or more, any seven or more, any eight or more, any nine or more, any ten or more, any eleven or more, any twelve or more, any thirteen or more, any fourteen or more, any fifteen or more, any sixteen or more, any seventeen or more, any eighteen or more, any nineteen or more, any twenty or more, any twenty-one or more, any twenty-two or more, any twenty-three or more, any twenty-four or more, any twenty-five or more, any twenty-six or more, any twenty-seven or more, any twenty-eight or more, any twenty-nine or more, or all thirty of VSIG4, ADAMTS2, NES, FAM107A, LEP, DAAM2, ARHGEF25, TIMP3, PRX, ALOX15B, HSPA12B, IGFBP5, CLEC4C, SLC9A3R2, ADAMTS1, SEMA3G, KRT5, AMPH, PRG2, PAPPA2, TEAD4, CRH, PITPNM3, TIMP4, PNMT, ZEB1, APOLD1, PLD4, CUX2, and HTRA4; or
(e) any one or more, any two or more, any three or more, any four or more, any five or more, any six or more, any seven or more, any eight or more, any nine or more, any ten or more, any eleven or more, any twelve or more, any thirteen or more, any fourteen or more, any fifteen or more, any sixteen or more, any seventeen or more, any eighteen or more, any nineteen or more, any twenty or more, any twenty-one or more, any twenty-two or more, any twenty-three or more, any twenty-four or more, any twenty-five or more, or all twenty-six of ADAMTS1, ADAMTS2, ALOX15B, AMPH, ARHGEF25, CELF4, DAAM2, FAM107A, HSPA12B, HTRA4, IGFBP5, KCNA5, KRT5, LCN6, LEP, LRRC26, NES, OLAH, PACSIN1, PAPPA2, PRX, PTGDR2, SEMA3G, SLC9A3R2, TIMP3, and VSIG4; or
(f) any one or more, any two or more, any three or more, any four or more, any five or more, any six or more, any seven or more, any eight or more, any nine or more, any ten or more, any eleven or more, any twelve or more, any thirteen or more, any fourteen or more, any fifteen or more, any sixteen or more, any seventeen or more, any eighteen or more, any nineteen or more, any twenty or more, any twenty-one or more, or all twenty-two of ADAMTS1, ADAMTS2, ALOX15B, ARHGEF25, CELF4, DAAM2, FAM107A, HTRA4, IGFBP5, KCNA5, KRT5, LCN6, LEP, LRRC26, NES, OLAH, PRX, PTGDR2, SEMA3G, SLC9A3R2, TIMP3, and VSIG4; or
(g) any one or more, any two or more, any three or more, any four or more, any five or more, any six or more, any seven or more, any eight or more, any nine or more, any ten or more, or all eleven of CLEC4C, ARHGEF25, ADAMTS2, LEP, ARRDC2, SKIL, PAPPA2, VSIG4, ARRDC4, CRH, and NES; or
(h) any one or more, any two or more, any three or more, any four or more, any five or more, any six or more, any seven or more, any eight or more, any nine or more, any ten or more, any eleven or more, any twelve or more, any thirteen or more, any fourteen or more, any fifteen or more, any sixteen or more, any seventeen or more, any eighteen or more, any nineteen or more, any twenty or more, any twenty-one or more, any twenty-two or more, any twenty-three or more, or all twenty-four of LEP, PAPPA2, KCNA5, ADAMTS2, MYOM3, ATP13A3, ARHGEF25, ADA, HTRA4, NES, CRH, ACY3, PLD4, SCT, NOX4, PACSIN1, SERPINF1, SKIL, SEMA3G, TIPARP, LRRC26, PHEX, LILRA4, and PER1; or
(i) any one or more, any two or more, any three or more, any four or more, any five or more, any six or more, any seven or more, any eight or more, any nine or more, any ten or more, any eleven or more, any twelve, any thirteen or more, any fourteen or more, any fifteen or more, any sixteen or more, any seventeen or more, any eighteen or more, any nineteen or more, any twenty or more, any twenty one or more, any twenty two or more, any twenty three or more, any twenty four or more, any twenty five or more, any twenty-six or more, any twenty-seven or more, any twenty-eight or more, any twenty-nine or more, any thirty or more, any thirty-one or more, any thirty-two or more, any thirty-three or more, any thirty-four or more, any thirty-five or more, any thirty-six or more, any thirty-seven or more, any thirty-eight or more, any thirty-nine or more, any forty or more, any forty-one or more, any forty-two or more, any forty-three or more, any forty-four or more, any forty-five or more, any forty-six or more, any forty-seven or more, any forty-eight or more, or all forth-nine of ADA, ADAMTS2, AKAP2, ARHGEF25, ARRB1, ARRDC2, ATOH8, CLEC4C, CPSF7, CUX2, FKBP5, FSTL3, GSTA3, HEG1, IGIP, INO80C, JAG1, JUN, KRT5, LEP, LILRA4, MRPS35, MSMP, NES, NFE2L1, NR4A2, NTRK2, PACSIN1, PER1, PLD4, PLEK, PRG2, RAP1GAP2, RGP1, SEMA3G, SH3PXD2A, SKIL, SMPD3, SPEG, SRPX, SYNPO, TEAD4, TIPARP, TNFRSF21, TPST1, TRPS1, UBE2Q1, VSIG4, and ZNF768; or
(j) any one or more, any two or more, any three or more, any four or more, any five or more, any six or more, any seven or more, any eight or more, any nine or more, any ten or more, any eleven or more, any twelve or more, or all thirteen of AKAP2, ARRB1, CPSF7, INO80C, JAG1, MSMP, NR4A2, PLEK, RAP1GAP2, SPEG, TRPS1, UBE2Q1, and ZNF768.

15. The method of claim 1, further comprising performing prenatal genetic screening testing or prenatal genetic diagnostic testing on a portion of the biosample obtained from a pregnant female.

16. The method of claim 1, further comprising providing the pregnant female with a therapeutic intervention for the treatment of preeclampsia selected from the group consisting of increased frequency of prenatal visits, antihypertensive medications to lower blood pressure, corticosteroid medications, anticonvulsant medications, bed rest, hospitalization, preterm delivery, cesarean delivery, induced labor, and combinations thereof and/or treating the subject pregnant human female with a low dose of aspirin, wherein a low dose of aspirin comprises about 50 to about 150 mg per day.

17. A circulating RNA (C-RNA) signature for preeclampsia or for an elevated risk of preeclampsia, the C-RNA signature comprising any one or more, any two or more, any three or more, or all four of HCG4P8, EYS, AGGF1P10, and GLYATL2.

18. A solid support array comprising a plurality of agents capable of binding and/or identifying a C-RNA signature of claim 17.

19. A kit comprising a plurality of probes capable of binding and/or identifying a C-RNA signature of claim 17.

20. A kit comprising a plurality of primers for selectively amplifying a C-RNA signature of claim 17.
